(19) United States
(12) Patent Application Publication
(10) Pub. No.: US 2023/0113185 A1
(43) Pub. Date: Apr. 13, 2023

(54) TRANSCATHETER MANIPULATION SYSTEMS, METHODS, AND DEVICES

(71) Applicant: W. L. Gore & Associates, Inc., Newark, DE (US)

(72) Inventors: Cody L. Hartman, Flagstaff, AZ (US); Edward E. Shaw, Flagstaff, AZ (US); Ryan S. Titone, Flagstaff, AZ (US)

(21) Appl. No.: 17/909,613

(22) PCT Filed: Mar. 2, 2021
(86) PCT No.: PCT/US2021/020438

§ 371 Date: Sep. 6, 2022

Related U.S. Application Data
(60) Provisional application No. 62/984,558, filed on Mar. 3, 2020.

(51) Int. Cl. A61F 2/24

(52) U.S. Cl. CPC A61F 2/2436; A61F 2/243

(57) ABSTRACT

A transcatheter system is disclosed, which includes a delivery sheath, a delivery system coupled to an implant and received by the delivery sheath, and a manipulation system. The manipulation system includes a manipulation device. The manipulation device includes an elongate portion that is extendable and retractable within the delivery sheath and an engaging portion extending from the elongate portion. The engaging portion is coupled to an internal bodily structure to be manipulated, and the manipulation system is received by the delivery sheath such that the engaging portion is extendable from the delivery sheath independently from the implant.

### CROSS-REFERENCE TO RELATED APPLICATION

This application is a national phase application of PCT Application No. PCT/US2021/020438, internationally filed on Mar. 2, 2021, which claims the benefit of Provisional Application No. 62/984,558, filed Mar. 3, 2020, which are incorporated herein by reference in their entireties for all purposes.

### BACKGROUND

Prosthetic valves and other medical implants have been developed to assist or replace native anatomical structure function. For example, bioprosthetic valves that implement flexible leaflets fabricated from biological tissue or synthetic materials may be implemented to augment or replace native valve function. Some conventional designs are configured to be implanted using a surgical approach that includes delivery to a target region within a patient's anatomy via open, surgical techniques. Other designs facilitate a transcatheter approach to implantation, which can offer advantages over surgical techniques. Transcatheter approaches to medical implant delivery are not without challenges. For example, prior to, during or after implantation anatomical features may interfere with implantation of an implant or implant function. And, the opposite may also be true—the implant, or process of implantation, may interfere with the proper functioning of anatomical features. Clearly, impediments in implantation, implant function, and anatomical function can be undesirable, and negatively impact a patient's health.

### SUMMARY

Various concepts addressed herein relate to manipulation systems that may be utilized in a transcatheter approach. Example systems are described that address manipulation of cardiac tissue, and specifically native valve leaflet material (e.g., anterior leaflets of the mitral valve), although a variety of implementations in a variety of anatomical locations are contemplated. For example, the manipulation systems may be used to manipulate other cardiac structures (e.g., the atrial septum or ventricular septum), vascular structures (e.g., vascular valves), respiratory structures (e.g., baffles, valves, or sphincters), gastrointestinal structures (e.g., esophageal sphincters), urinary tract structures (e.g., urinary tract valves), and others. Moreover, though transcatheter techniques are provided by way of primary examples, other examples include percutaneous and laparoscopic approaches, among others.

According to one example (“Example 1”), a transcatheter system includes a delivery sheath, a delivery system coupled to an implant, where the delivery system is received by the delivery sheath, and a manipulation system including a manipulation device. The manipulation device includes an elongate portion that is extendable and retractable within the delivery sheath and an engaging portion extending from the elongate portion, the engaging portion being configured to couple to an internal bodily structure to be manipulated. The manipulation system is also received by the delivery sheath such that the engaging portion is extendable from the delivery sheath independently from the implant.

According to another example further to Example 1 (“Example 2”), the delivery sheath is configured to receive the manipulation system and the implant such that the manipulation system is positioned adjacent the implant within the delivery sheath.

According to another example further to Example 1 or 2 (“Example 3”), the elongate portion and the engaging portion are integrally formed.

According to another example further to any one of Examples 1 to 3 (“Example 4”), the engaging portion is configured to receive an anterior mitral leaflet.

According to another example further to any one of Examples 1 to 3 (“Example 5”), the engaging portion is configured to puncture an anterior mitral leaflet.

According to another example further to any one of Examples 1 to 5 (“Example 6”), the engaging portion includes a curved section having a radius of curvature.

According to another example further to any one of Examples 1 to 6 (“Example 7”), the engaging portion includes a recurved hook configuration.

According to another example further to any one of Examples 1 to 7 (“Example 8”), the elongate portion and the engaging portion are formed of an elastic filament material.

According to another example further to any one of Examples 1 to 8 (“Example 9”), the manipulation system includes a constraining sheath receiving the elongate portion and the engaging portion of the manipulation device in an undeployed configuration.

According to another example further to any one of Examples 1, 2 or, 4 to 9 (“Example 10”), the engaging portion includes a coupling section, a piercing section, and an anchoring section disposed between the coupling section and the piercing section.

According to another example further to Examples 10 (“Example 11”), the coupling section is releasably coupled with the elongate portion of the manipulation system.

According to another example further to any one of Examples 1 to 11 (“Example 12”), the engaging portion is configured to elastically transition from a compact, delivery configuration to an expanded, deployed configuration.

According to another example (“Example 13”), a transcatheter system includes a delivery system for an implant and a manipulation system integrated with the delivery system, the delivery system and the manipulation system being independently controllable.

According to another example further to Example 13 (“Example 14”), the implant is a prosthetic valve and the manipulation system is configured to anchor to and retract a native valve leaflet.

According to another example further to Example 13 or 14 (“Example 15”), the transcatheter system further includes a delivery sheath configured to position the delivery system adjacent the manipulation system at a treatment site in a body of a patient.

According to another example further to one of Examples 13 to 15 (“Example 16”), the transcatheter system further includes a handle assembly configured to independently control each of the delivery system and the manipulation system.

According to another example (“Example 17”), a method of delivering an implant (e.g., a prosthetic valve) to a treatment site (e.g., within a native valve orifice) includes: positioning a manipulation system at the treatment site; engaging and retracting, using the manipulation system, tissue (e.g., a leaflet of the native valve orifice); positioning, using a delivery catheter, an implant at the treatment site; and implanting the implant such that the tissue (e.g., leaflet) is captured between the implant and tissue at the treatment site (e.g., native valve orifice).

According to another example further to Example 17 (“Example 18”), the method further includes retracting the manipulation system after expanding the implant.

According to another example further to Example 17 or 18 (“Example 19”), the manipulation system includes an elongate portion and an engaging portion integral with the elongate portion, the engaging portion having at least one curved section configured to engage the tissue (e.g., leaflet).

According to another example further to Example 19 (“Example 20”), retracting the manipulation system after implanting the implant includes straightening the engaging portion.

According to another example further to Example 20 (“Example 21”), straightening the engaging portion comprises extending a constraining sheath along the manipulation system.

While multiple embodiments are disclosed, still other embodiments will become apparent to those skilled in the art from the following detailed description, which shows and describes illustrative examples. Accordingly, the drawings and detailed description are to be regarded as illustrative in nature and not restrictive.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A through 1D are partial side views of different transcatheter system configurations, according to some embodiments;

FIG. 1E is a schematic diagram of a portion of a manipulation system, according to some embodiments;

FIG. 1F is a schematic diagram of selected transcatheter system components, according to some embodiments;

FIG. 2A is a perspective view of an engaging portion of a manipulation device in a restricted, or collapsed configuration, according to some embodiments;

FIG. 2B is a perspective view of an engaging portion of a manipulation device in an expanded, or deployed configuration, according to some embodiments;

FIG. 2C is a perspective view of another engaging portion of a manipulation device in an expanded configuration, according to some embodiments;

FIG. 2D is a perspective view another engaging portion of a manipulation device in an expanded configuration, according to some embodiments;

FIGS. 3A to 3E illustrate a tissue manipulation sequence, according to some embodiments;

FIGS. 4A and 4B show a configuration of an engaging portion of a manipulation device, according to some embodiments;

FIGS. 4C and 4D show a configuration of an engaging portion of a manipulation device, according to some embodiments;

FIGS. 5A through 5D show a deployment sequence and retrieval operation using a manipulation system, according to some embodiments;

FIG. 6 shows a delivery system, according to some embodiments;

FIGS. 7A and 7B show a prosthetic valve, according to some embodiments;

FIGS. 8A through 8C show additional examples of prosthetic valves, according to some embodiments;

FIGS. 8D and 8E shows another example of a prosthetic valve, according to some embodiments;

FIG. 8F shows another example of a prosthetic valve, according to some embodiments; and

FIG. 9 shows a flow diagram of a method of delivering a prosthetic valve, according to some embodiments.

### DETAILED DESCRIPTION

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### Definitions and Terminology

A “prosthetic valve” (also referred to as a bioprosthetic valves) may be configured to replace a native valve, such as any of the cardiac valves (e.g., aortic, mitral, or tri-cuspid) or other bodily valves (e.g., vascular valves). Such prosthetic valves may include leaflets that are flexible and fabricated from biological tissue, synthetic materials, or combinations thereof. In some prosthetic valve designs, the leaflets are coupled onto a relatively more rigid frame that supports the leaflets and provides dimensional stability when implanted. Typically, the leaflets move under the influence of fluid pressure where, in operation, the leaflets open when the upstream fluid pressure exceeds the downstream fluid pressure and close when the downstream fluid pressure exceeds the upstream fluid pressure. The free edges of the leaflets generally coapt under the influence of the downstream fluid pressure, which closes the valve to prevent downstream blood from flowing retrograde through the valve. In turn, the free edges of the leaflets separate, or move away from one another under the influence of upstream fluid pressure to permit flow antegrade through the valve.

The terms “native valve orifice” and “tissue orifice” as used herein refer to an anatomical structure into which a prosthetic valve can be placed. Such anatomical structures include, but are not limited to, a location wherein a cardiac valve may or may not have been surgically removed. It is understood that other anatomical structures that can receive a prosthetic valve include, but are not limited to, veins, arteries, ducts and shunts. It is further understood that a tissue orifice or implant site may also refer to a location in a synthetic or biological conduit that may receive a valve.

The term “membrane” as used herein refers to a sheet of material comprising a single composition, such as, but not limited to, expanded fluoropolymer.

The term “composite material” as used herein refers to a material including two or more material components with one or more different material properties from the other. In some examples, a composite material includes at least a first material component in the form of a membrane and a second material component in the form of a polymer that is combined with the membrane (e.g., by coating and/or imbibing processes). The term “laminate” as used herein refers to multiple layers of membrane, composite material, or other materials, such as, but not limited to a polymer, such as, but not limited to an elastomer, elastomeric or non-elastomeric material, and combinations thereof.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material as is generally known.

The term “film” as used herein generically refers to one or more of the membrane, composite material, or laminate.

As used herein, “couple” means join, connect, attach, adhere, affix, or bond, whether directly or indirectly, and whether permanently or temporarily.

The term “leaflet” or “leaflet construct”, which comprises a plurality of leaflets, as used herein in the context of prosthetic valves is a component of a one-way valve wherein the leaflet is operable to move between an open and closed position under the influence of a pressure differential. In an open position, the leaflet allows fluid (e.g., blood) to flow through the valve. In a closed position, the leaflet substantially blocks retrograde flow through the valve by occluding the valve orifice. In embodiments comprising multiple leaflets, each leaflet cooperates with at least one neighboring leaflet or secondary structure to block the retrograde flow of blood. The pressure differential in the blood is caused, for example, by the contraction of a ventricle or atrium of the heart, such pressure differential typically resulting from a fluid pressure building up on one side of the leaflets when closed, for example, by the contraction of a ventricle or atrium of the heart. As the pressure on an inflow side of the valve rises above the pressure on the outflow side of the valve, the leaflets open and blood flows therethrough. As blood flows through the valve into a neighboring chamber or blood vessel, the pressure on the inflow side equalizes with the pressure on the outflow side. As the pressure on the outflow side of the valve rises above the blood pressure on the inflow side of the valve, the leaflet returns to the closed position generally preventing retrograde flow of blood through the valve.

It is appreciated that leaflets, where not required by the specific design or mode of function of the disclosed embodiment, may be rigid such as in mechanical valves or may be flexible as in bioprosthetic and synthetic valves. It is further appreciated that, although embodiments provided herein include a frame that supports the leaflets, the leaflets may not necessarily be supported by a frame. In other embodiments, the leaflets may be constructed as in the tissue valve art that are formed into the desired shape without a frame.

The term “manipulate” or “manipulation” includes grasping, positioning, controlling, retracting, extending, moving, or otherwise forcibly maintaining or changing physical position.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example.

Certain relative terminology is used to indicate the relative position of components and features. For example, words such as “top”, “bottom”, “upper,” “lower,” “left,” “right,” “horizontal,” “vertical,” “upward,” and “downward” are used in a relational sense (e.g., how components and features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

### Description

Various concepts and illustrative examples of this description relate to manipulation systems deployable in a transcatheter approach, or similar approach, separately from or in association with delivery of a medical implant, also described as an implantable device, or simply an implant. The manipulation systems are configured to position, control, retract, move or otherwise physically manipulate internal body structures, such as body tissue (e.g., heart valve leaflet structures) via a transcatheter approach. In some examples, the manipulation systems form part of a transcatheter system that also includes a delivery system for an implant. In the description that follows, various examples are provided in the context of the implant being a prosthetic valve. The inventive concepts addressed herein are not so limited. The implant (and the associated delivery system) may include any of a variety of medical devices, including any prosthetic valve, graft, stent, stent graft, filter, conduit, or any other device of similar structure and/or function.

With the foregoing in mind, the figures show various components and configurations fora transcatheter system 1000 according to some examples. As shown, the transcatheter system 1000 may include an implant (e.g., prosthetic valve 1001) and a delivery system 1002 for the implant (e.g., prosthetic valve 1001), along with a manipulation system 1003 adapted for manipulating bodily structures (e.g., tissue) through a transcatheter approach. In some examples, the transcatheter system 1000 includes a delivery sheath 1504 that serves as a common mechanism for positioning the delivery system 1002 (along with the implant, e.g., prosthetic valve 1001) and the manipulation system 1003 at a desired treatment site. Thus, the system components may be positioned at the desired treatment site using a common system that includes a sheath or similar device having one or more lumens configured to accommodate each of the delivery system 1002 and the manipulation system 1003. Alternatively, the manipulation system 1003 may be utilized separately (e.g., with the delivery system 1002 not included as part of the transcatheter system 1000).

The various components of the transcatheter system 1000 (e.g., the delivery sheath 1504) may be formed of a variety of biocompatible materials commonly associated with transcatheter systems. For example, the sheath and constraint components may include any of a variety of biocompatible metallic and polymeric materials, such as polyurethanes, polyethylenes (e.g., UHMWPE), fluoropolymers (e.g., polytetrafluoroethylene (PTFE) and expanded PTFE (ePTFE)) and others.

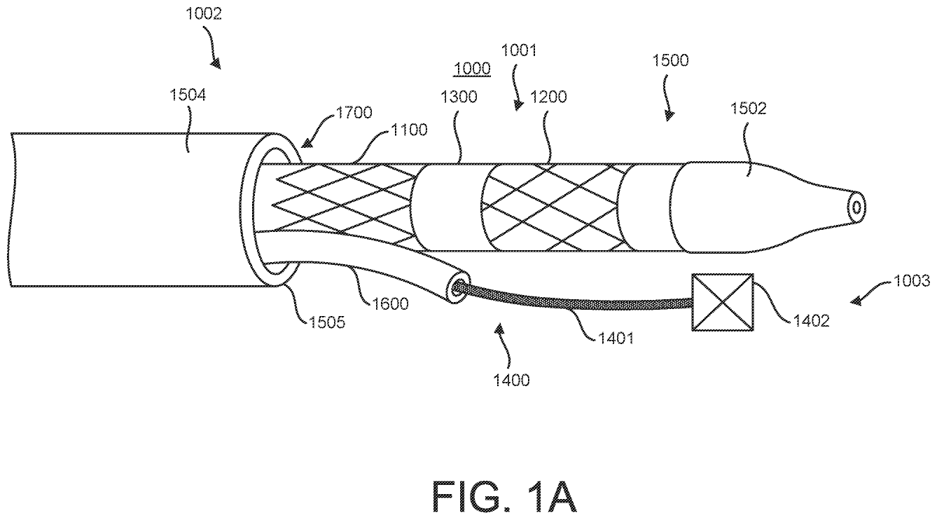

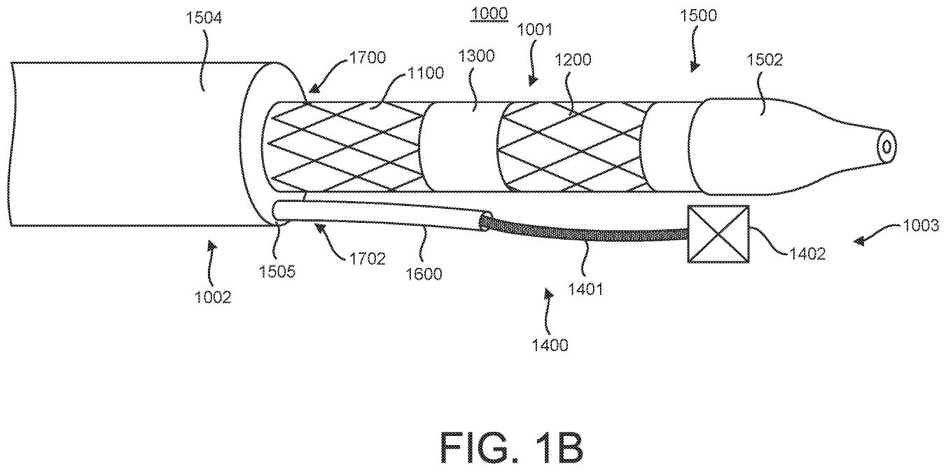

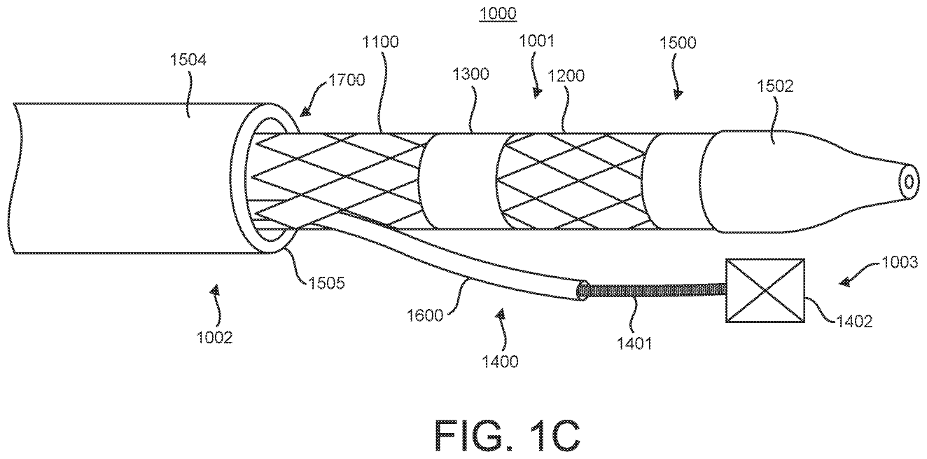

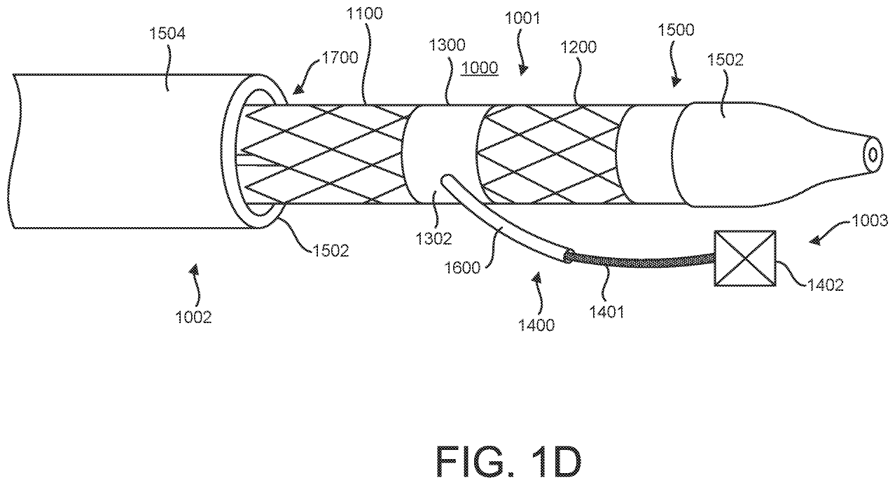

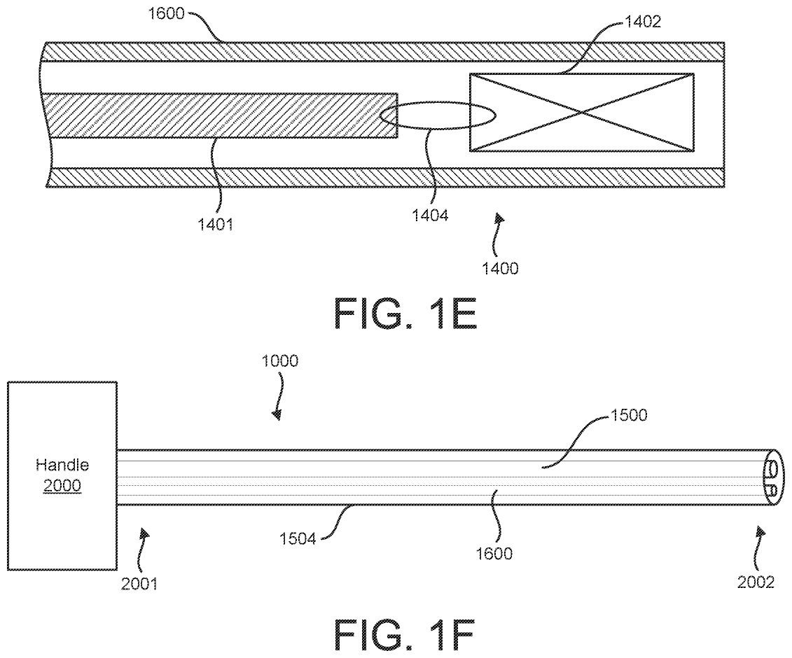

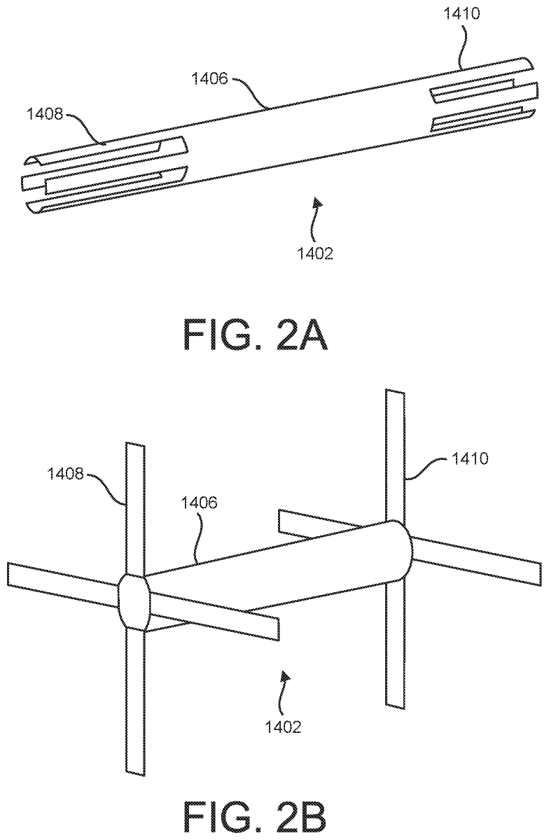

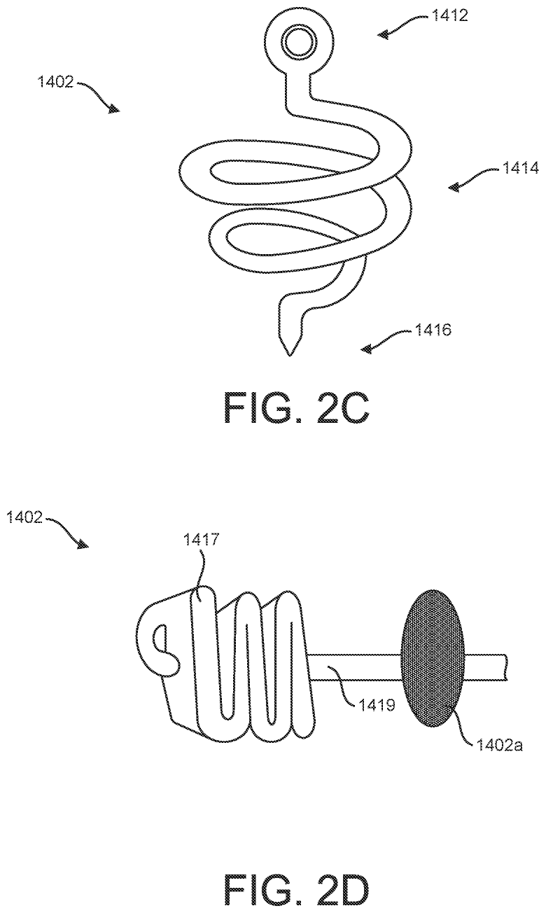

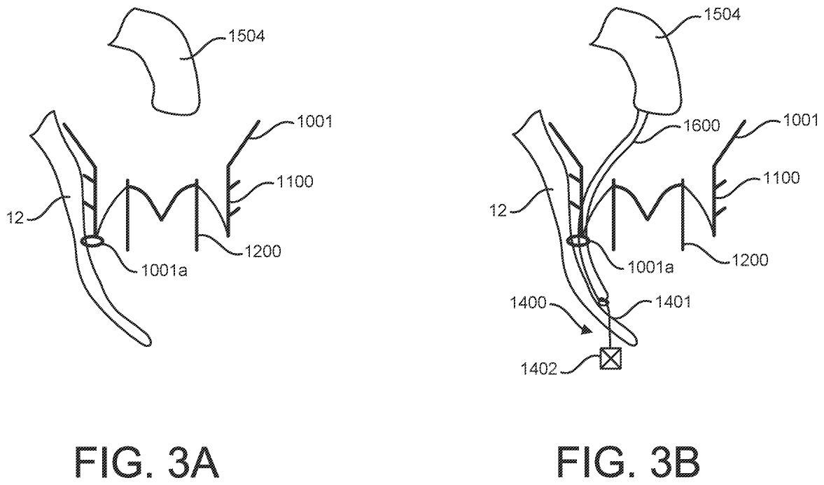

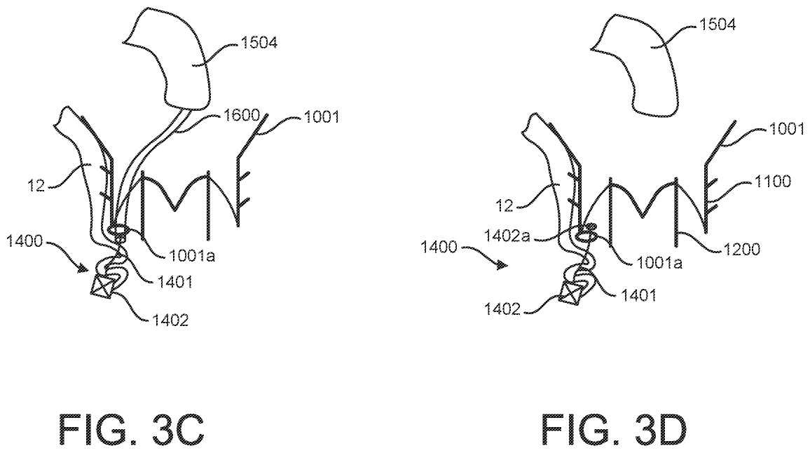

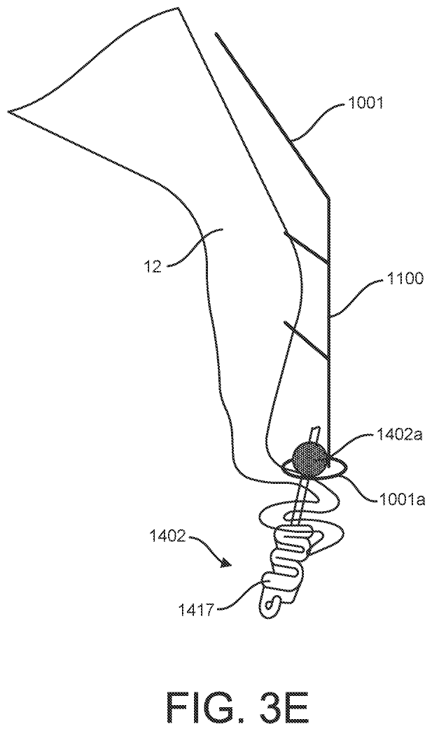

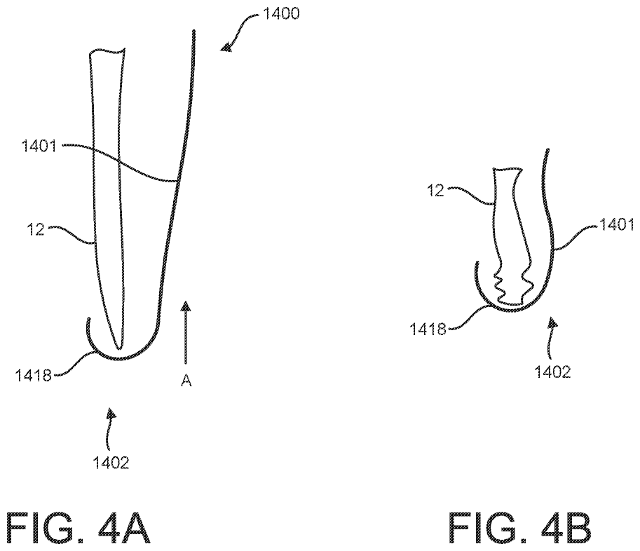

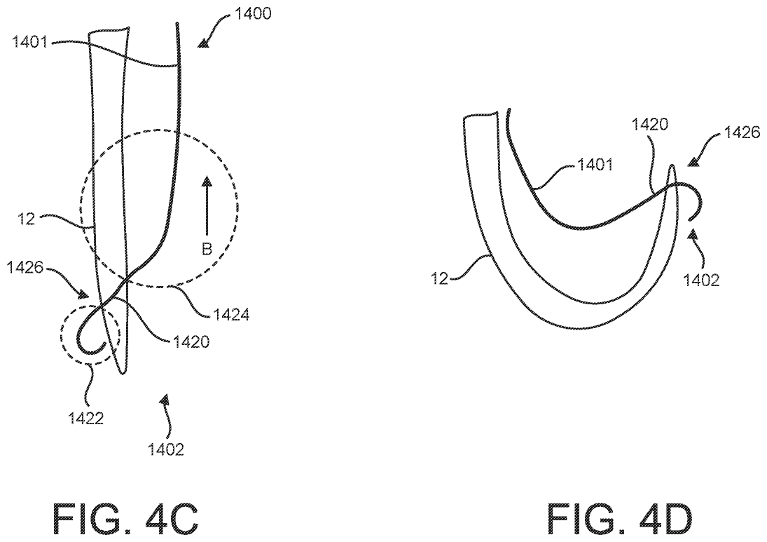

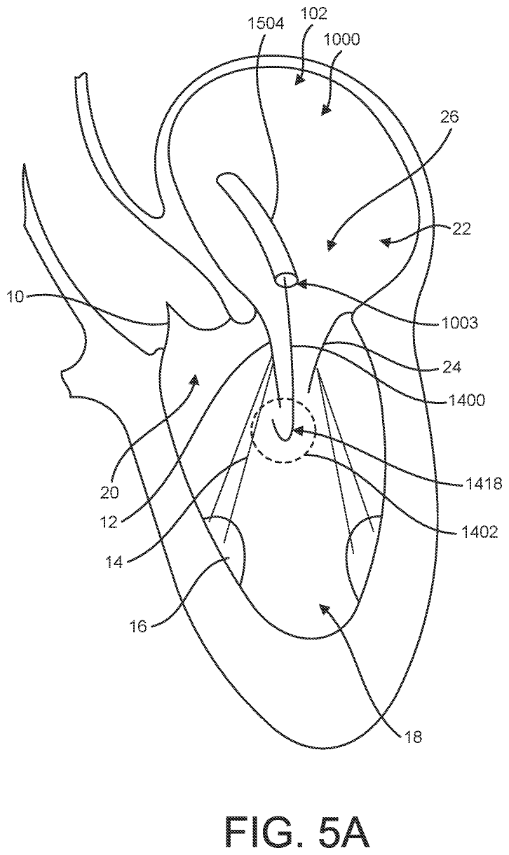

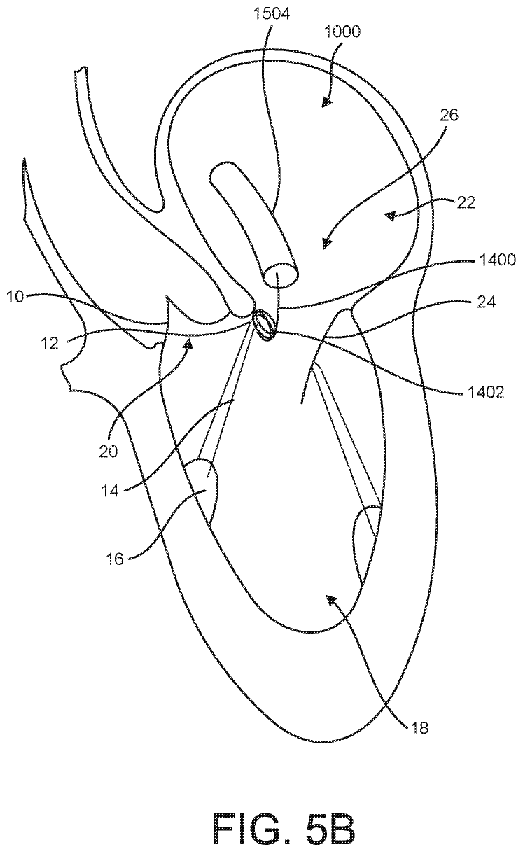

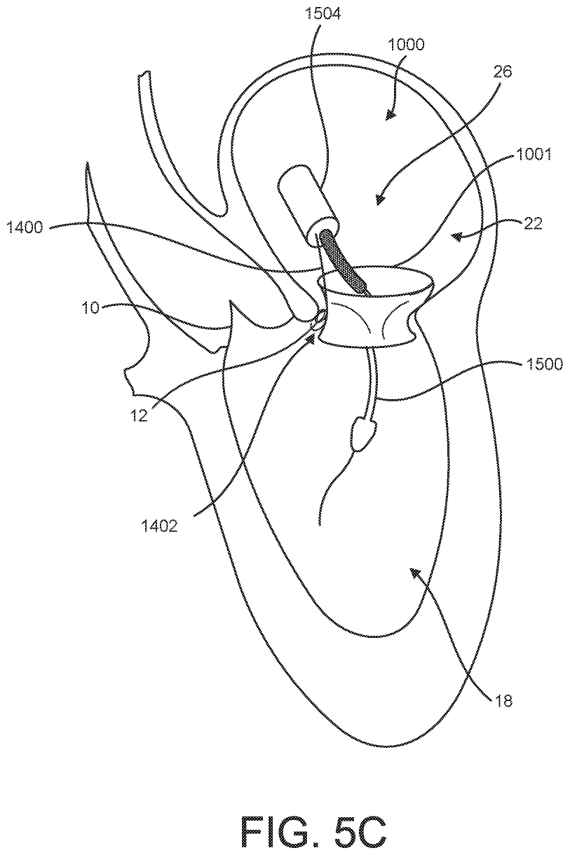

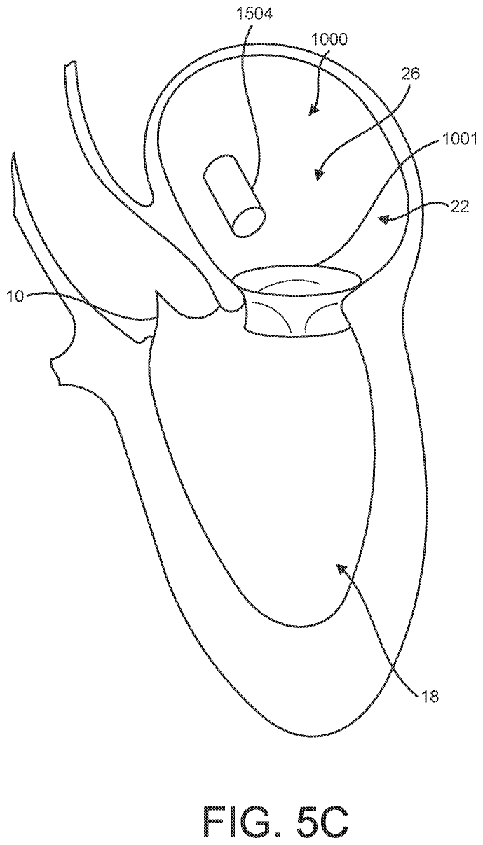

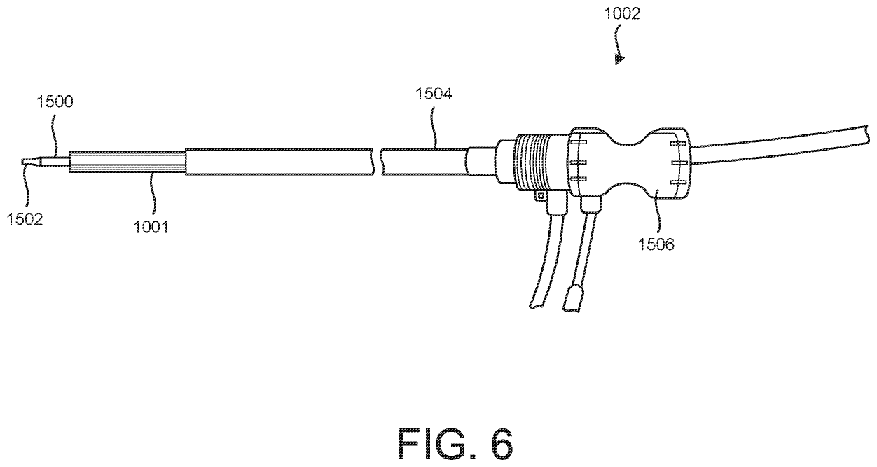

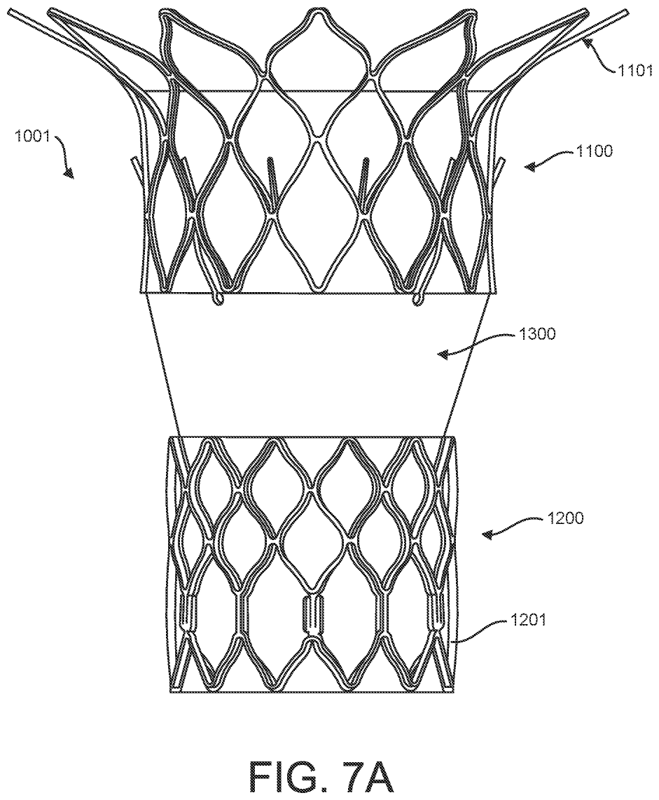

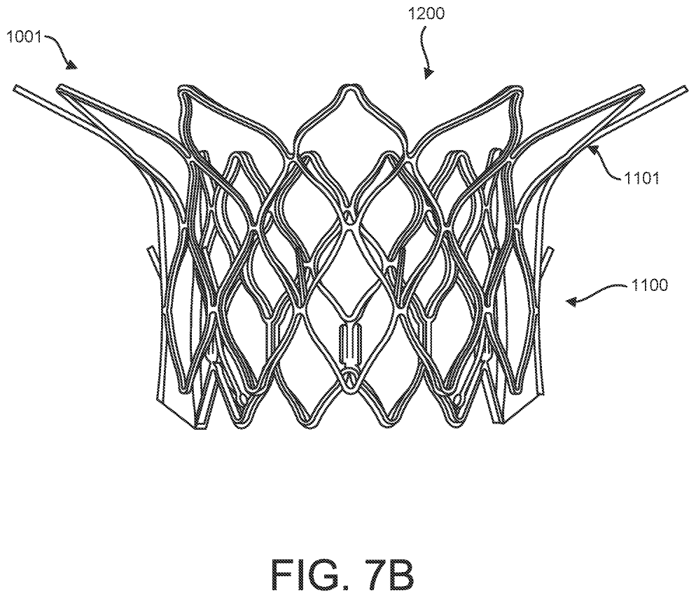

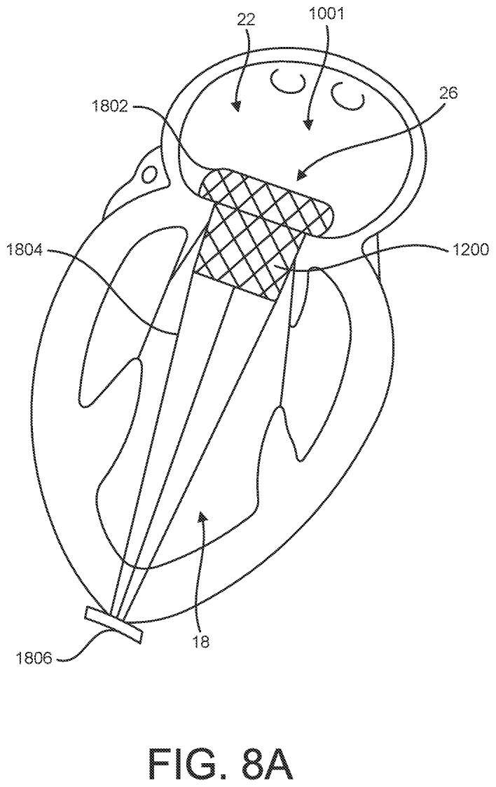

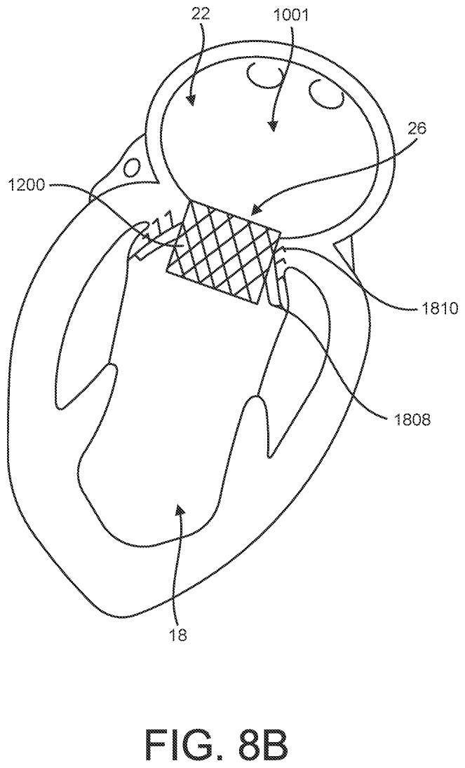

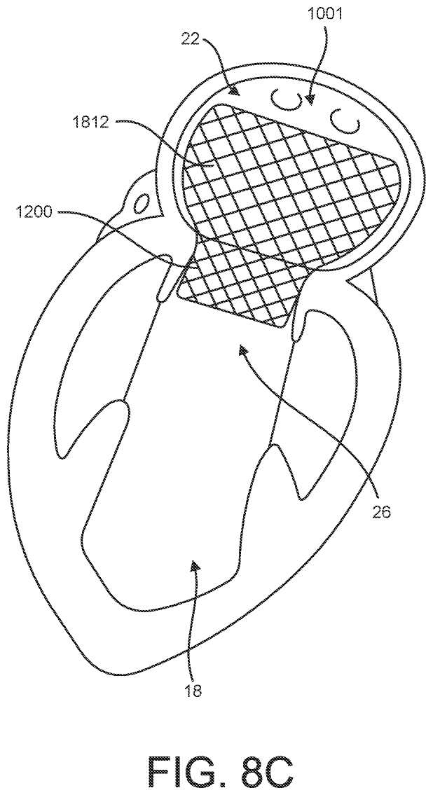

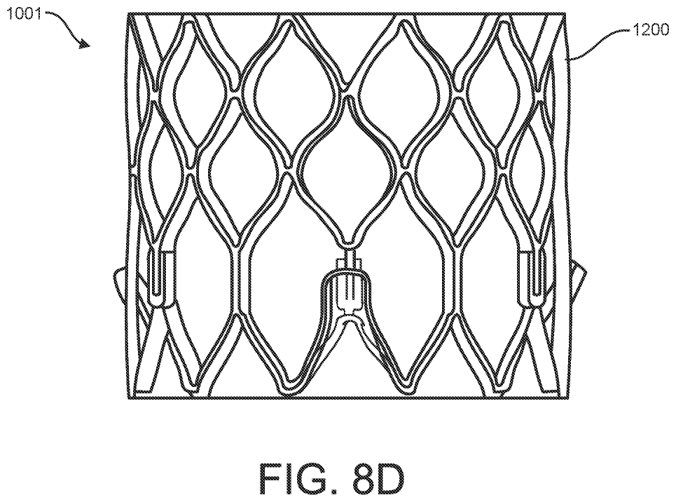

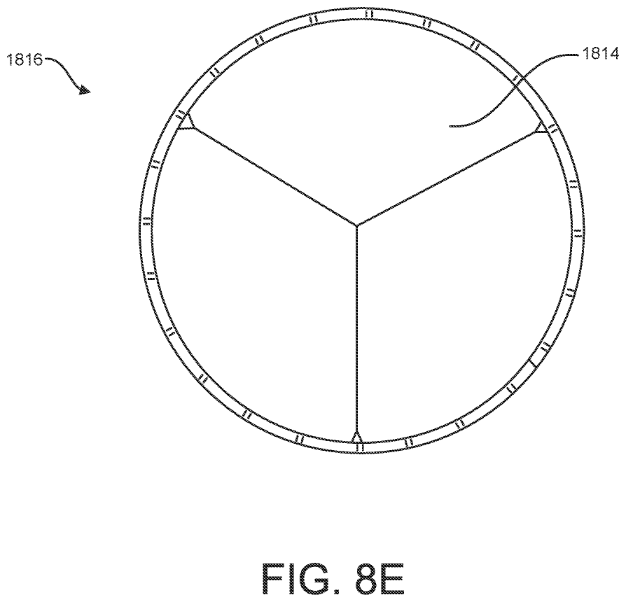

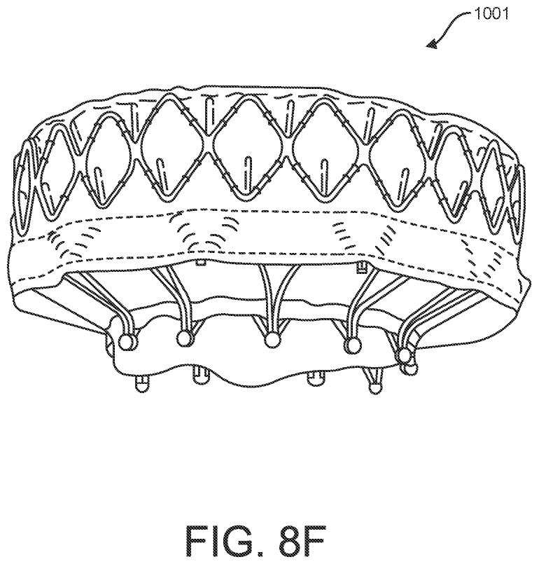

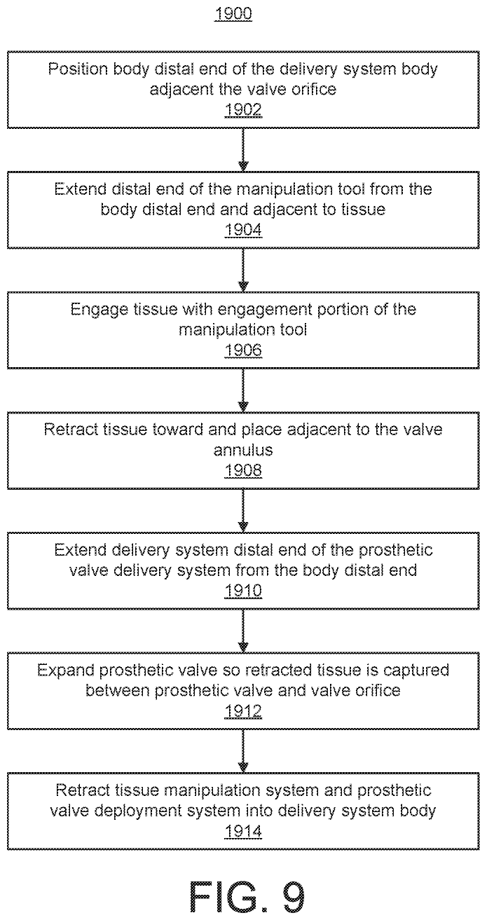

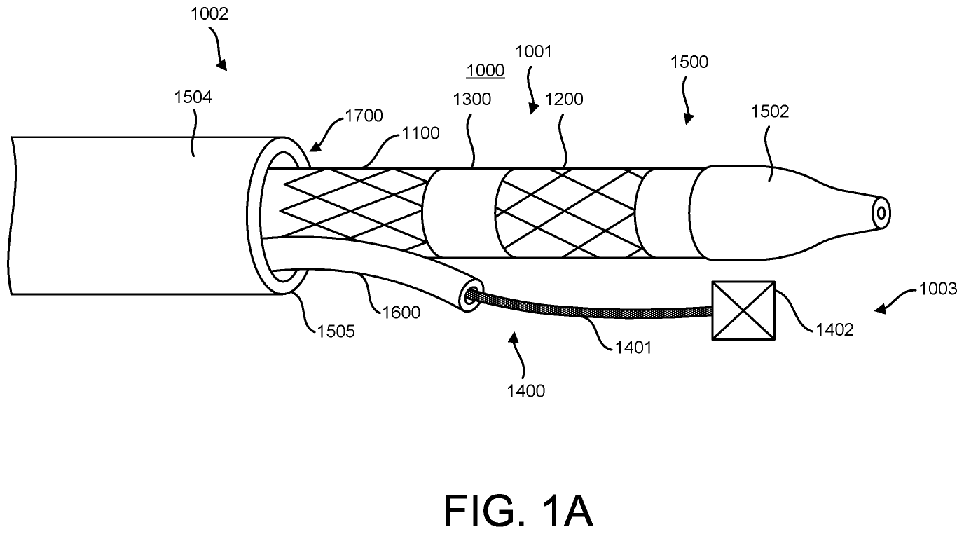

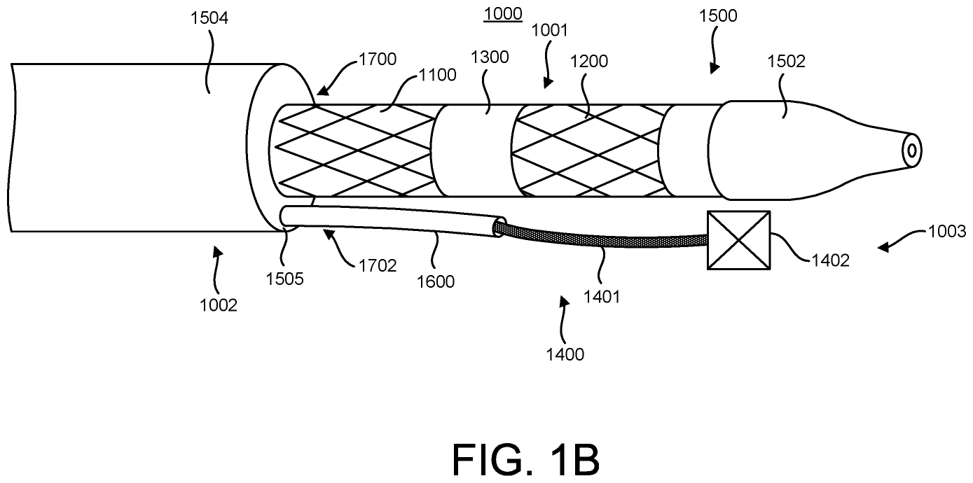

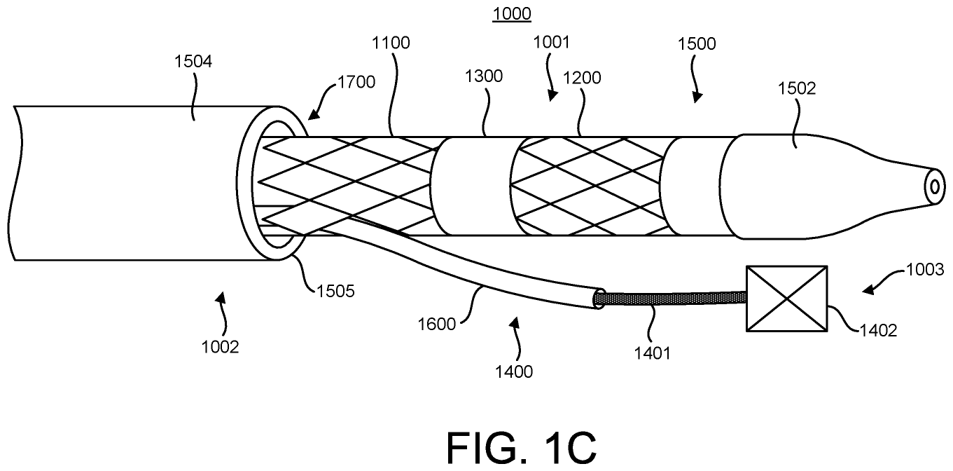

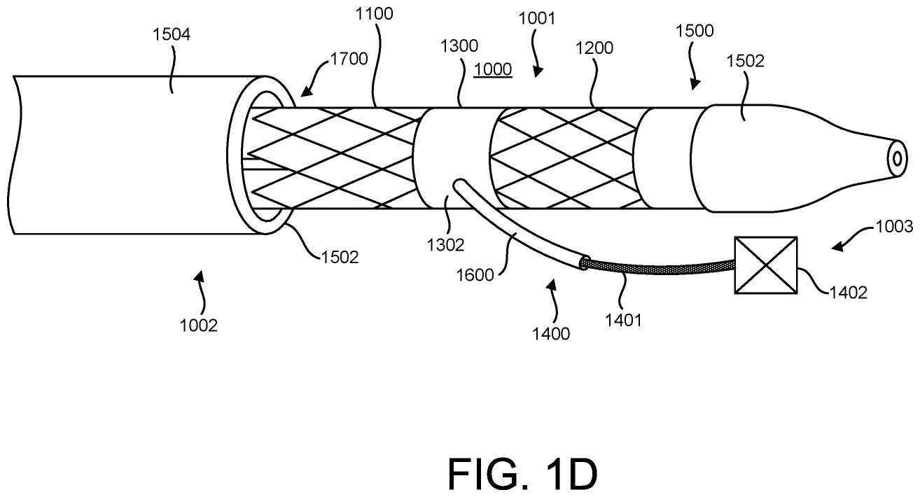

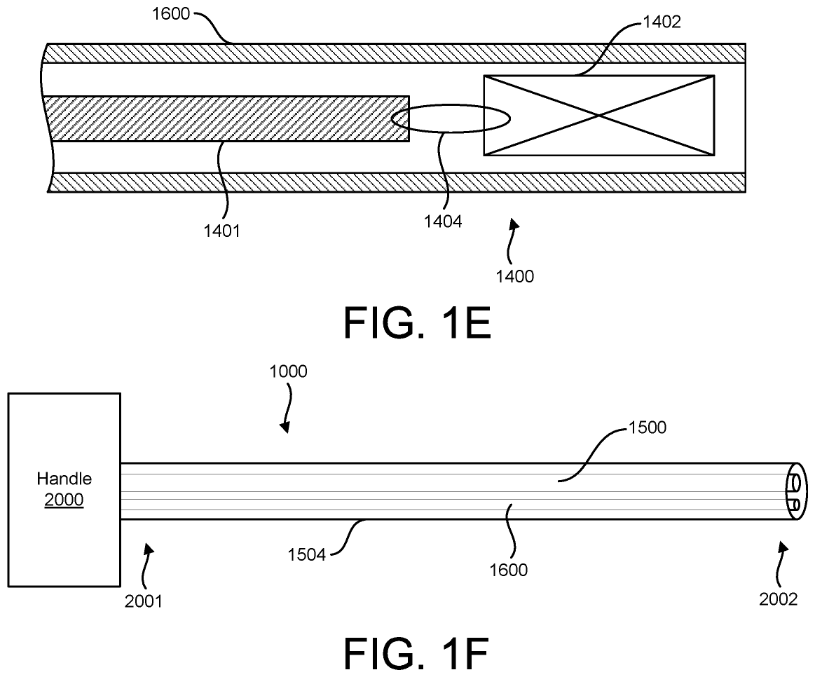

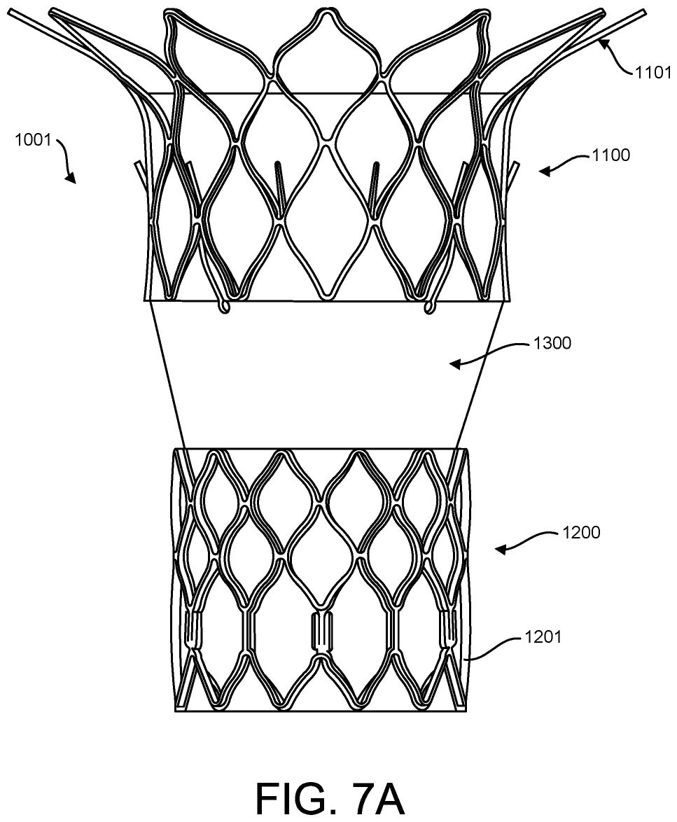

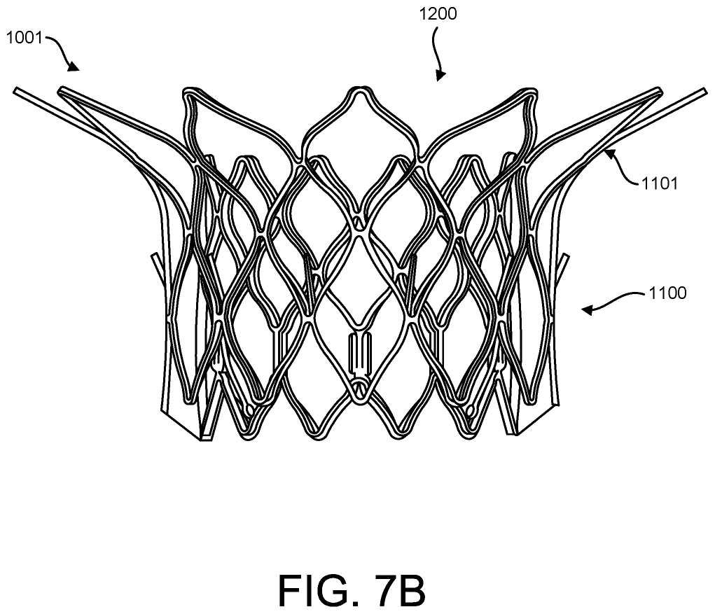

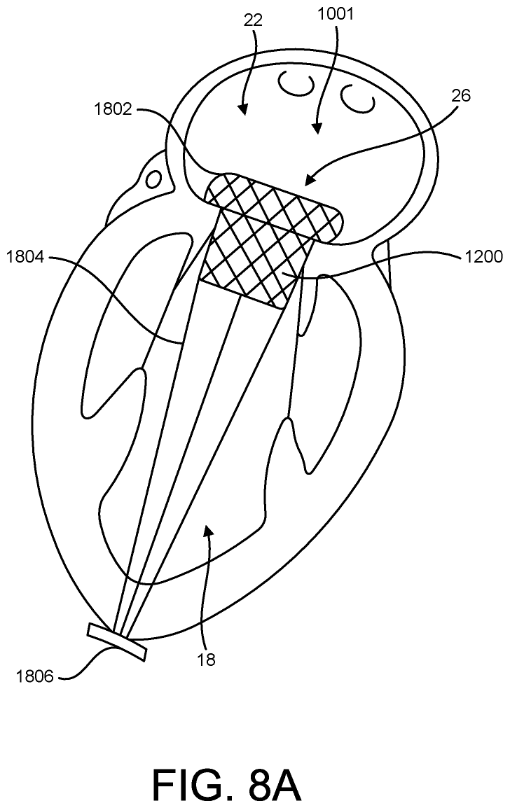

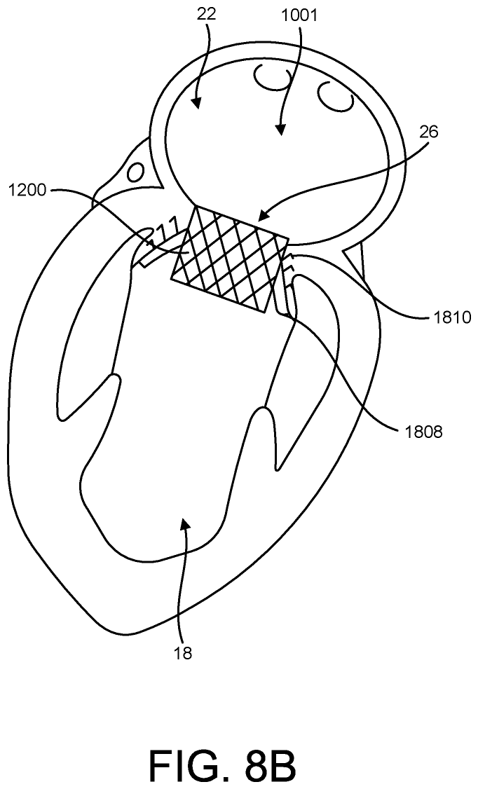

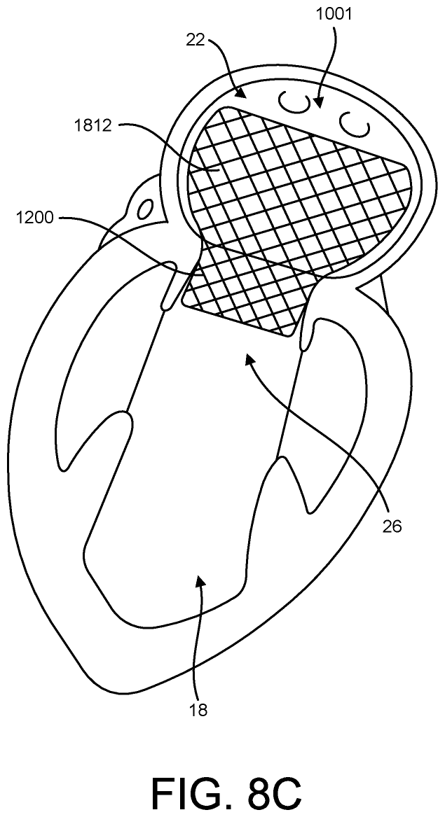

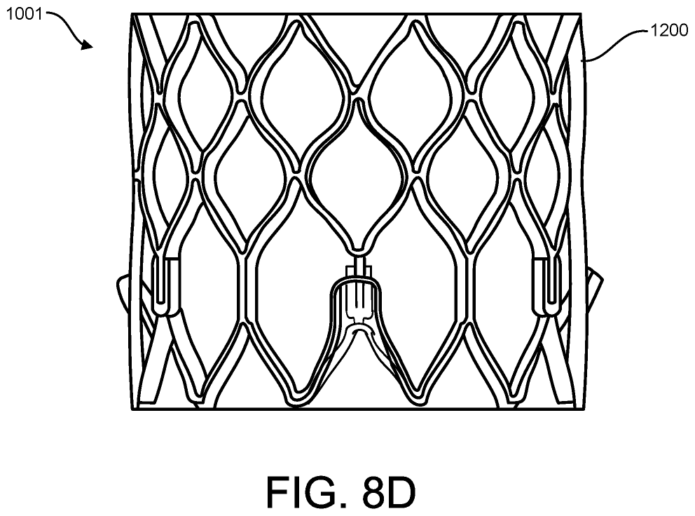

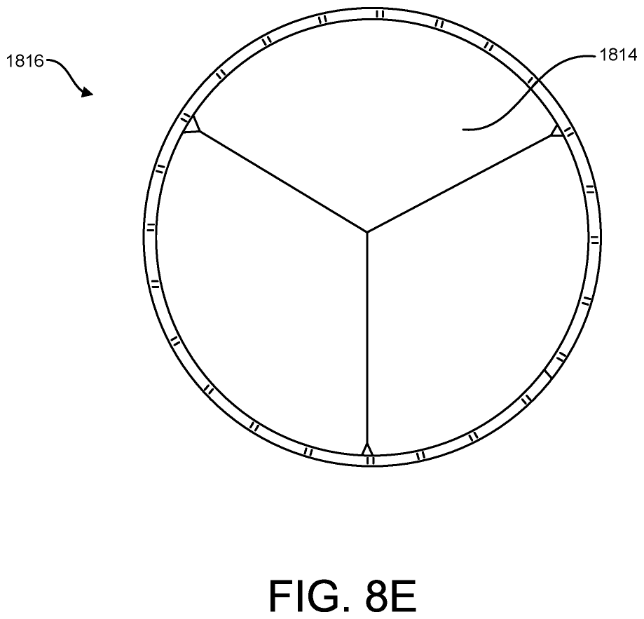

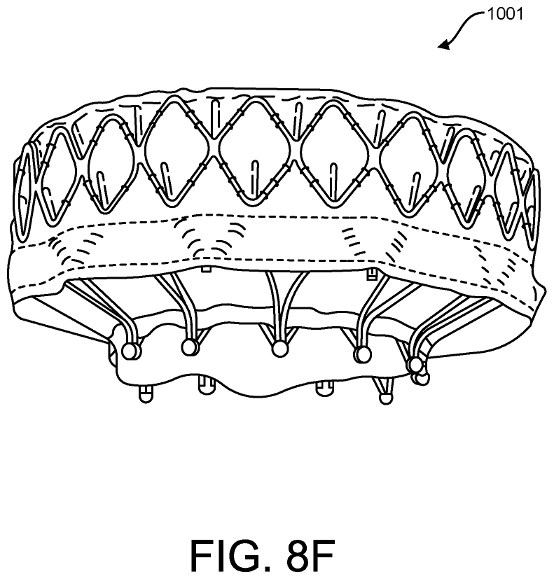

FIGS. 1A through 1D each show a transcatheter system 1000 which includes an implant (e.g., prosthetic valve 1001), a delivery system 1002 for the implant (e.g., prosthetic valve 1001), and a manipulation system 1003, according to some embodiments. As discussed, the implant (e.g., prosthetic valve 1001) may include various implants, but in the figures is illustrated as a prosthetic valve. The implant (e.g., prosthetic valve 1001) may be coupled to and carried by a delivery system 1002 (e.g., a catheter) in a predeployed, or delivery configuration. For ease of reference, implant (e.g., prosthetic valve 1001) is referred to interchangeably as prosthetic valve 1001, but it is to be understood that this is to be taken in an illustrative, not a limiting sense. It should also be clear that, though a multi-part frame prosthetic valve design is shown in FIGS. 1A through 1D and 7A and 7B, other valve designs may be implemented, such as those shown in FIGS. 8A to 8E, for example.

With the foregoing in mind, the prosthetic valve 1001 in FIGS. 1A-1D includes an anchor frame subcomponent 1100 and a leaflet frame subcomponent 1200 that are longitudinally offset from one another (also referred to as being delivered in series) and coupled together with an interstage 1300 therebetween. In some examples, the prosthetic valve 1001 may include the leaflet frame subcomponent 1200 while omitting the anchor frame subcomponent 1100 and the interstage 1300. In some examples, the anchor frame subcomponent 1100 and the interstage 1300 may be pre-nested within the leaflet frame subcomponent 1200 prior to delivery and deployment of the prosthetic valve 1001.

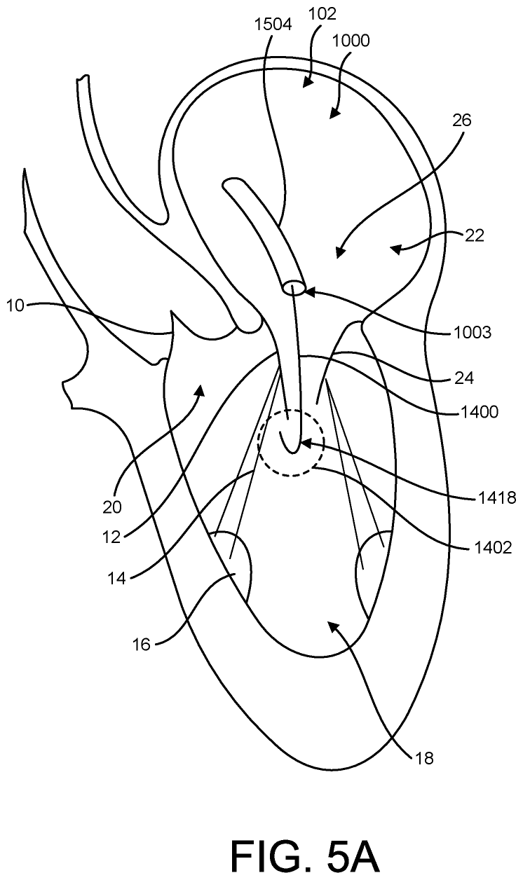

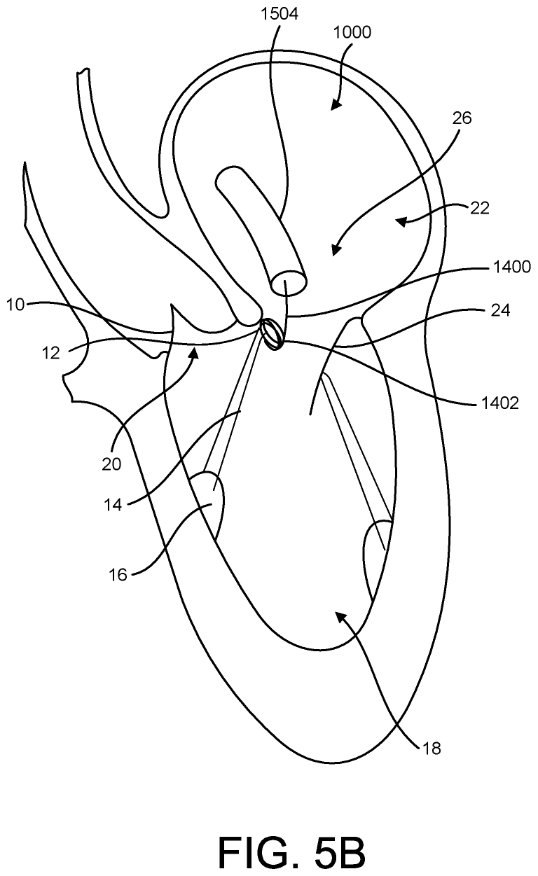

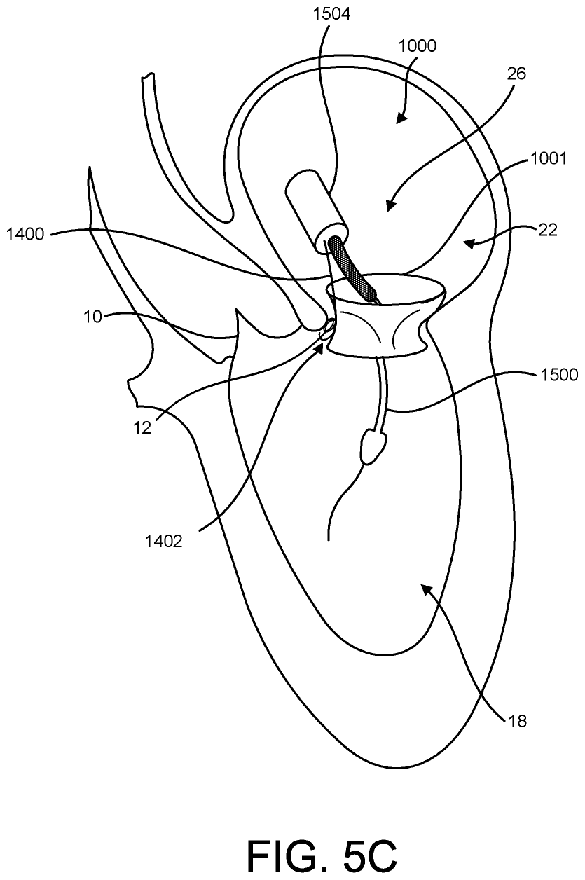

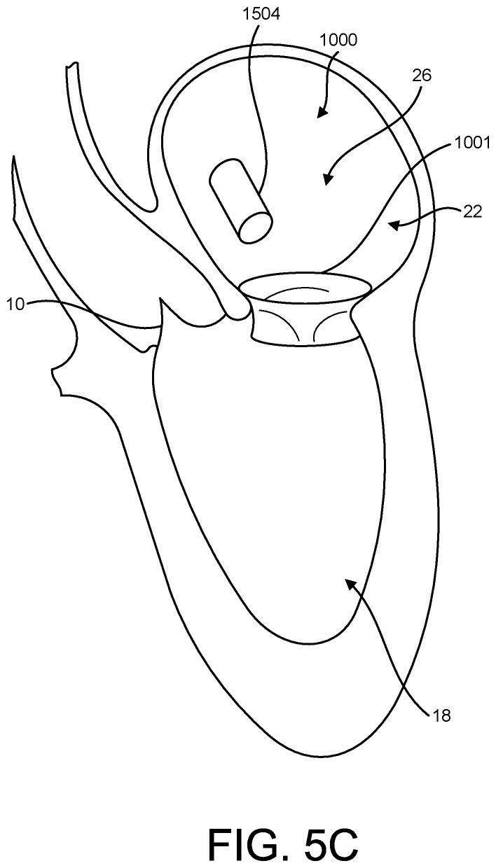

As shown, the delivery system 1002 is operable to position and deploy the implant (e.g., prosthetic valve 1001), the prosthetic valve 1001 in FIGS. 1A-1D, at a desired treatment site in a body of a patient, such as within a tissue orifice (a native valve orifice 26 as illustrated in FIGS. 5A through 5D). The delivery system 1002 is configured as appropriate for the implant-type to be associated with the delivery system 1002. For example, the delivery system 1002 may be a prosthetic valve delivery system (e.g., self-expanding or expandable), stent graft delivery system (e.g., self-expanding or expandable), or suited for use with any of the other previously-described implant examples.

The manipulation system 1003 is configured for use to manipulate tissue, such as tissue adjacent the implant (e.g., prosthetic valve 1001), during positioning and deployment of the implant (e.g., prosthetic valve 1001). In other examples, the manipulation system 1003 may additionally or alternatively be used to manipulate other bodily structures and features, such as other implants, blockages (e.g., thrombi or calcification), or others.

As referenced, both the delivery system 1002 (e.g., a prosthetic valve delivery system) and the manipulation system 1003 (e.g., tissue manipulation system) may be contained within a transcatheter system 1000 as an integrated unit. For example, the delivery system 1002 and the manipulation system 1003 may each be maintained together within a portion of the transcatheter system 1000.

In some embodiments, the delivery system 1002 includes a delivery device 1500 (e.g., a delivery catheter shaft) and the transcatheter system 1000 includes a delivery sheath 1504 that is an elongated tubular member having one or more internal lumens. For reference, in the illustrations of FIGS. 1A to 1D, the delivery system 1002 and the manipulation system 1003 are shown distally extended from the delivery sheath 1504 of the transcatheter system 1000.

In use, the delivery system 1002 and the manipulation system 1003 may be retracted into the delivery sheath 1504 during transcatheter delivery to a desired treatment site. For reference purposes, and though a variety of diametric profiles are contemplated, in some examples, a maximum diametric profile of the delivery device 1500 is twenty-four French (24F) or less.

In some examples, as shown in the embodiment of FIG. 1A, the delivery sheath 1504 has a first lumen 1700 through which the delivery device 1500 passes, and which allows movement of the delivery device 1500 of the delivery system 1002 in the proximal and distal directions with respect to the delivery sheath 1504. Additionally, the first lumen 1700 also receives the manipulation system 1003 such that portions of the delivery system 1002 and the manipulation system 1003 are positioned next to one another inside the first lumen 1700. The first lumen 1700 may include features to facilitate sliding of portions of the delivery system 1002 and the manipulation system 1003 within the delivery sheath 1504. Such features may include a lubricious layer, for example one or more layers of material with a low coefficient of friction or lubricant (e.g., a hydrogel) that facilitates smooth translation within the first lumen 1700.

In some examples, as shown in the embodiment of FIG. 1B, the delivery sheath 1504 has a plurality of lumens, including a second lumen 1702 in addition to the first lumen 1700. As such, the first lumen 1700 receives a portion of the delivery system 1002 and may assist with constraining the implant (e.g., prosthetic valve 1001). In various examples the second lumen 1702 receives a portion of the manipulation system 1003 such that, as constrained inside the first and second lumens 1700 and 1702, the portions of the delivery system 1002 and the manipulation system 1003 do not directly come into contact with one other, or such contact is reduced, thereby reducing interference between the systems during their respective sliding in the first lumen 1700 and the second lumen 1702. Additionally, the first and second lumens 1700 and 1702 may have different sizes, shapes, and configurations. For example, the first and second lumens 1700 and 1702 may have a circular, ovular, polygonal, or any other suitable cross-sections, as well as cross-sectional areas. In some examples, there may be additional lumens extending through the length of the delivery sheath 1504, as suitable.

In various embodiments, the manipulation system 1003 includes a manipulation device 1400 and a constraining sheath 1600, where the manipulation device 1400 is slidably received inside the constraining sheath 1600 such that the manipulation device 1400 may be extended from and retracted into the constraining sheath 1600. The constraining sheath 1600 and the manipulation device 1400 are, in turn, positioned within the delivery sheath 1504 and are extendable from a distal end 1505 of the delivery sheath 1504. In some examples, the delivery sheath 1504 is configured to receive the manipulation device 1400 and the constraining sheath 1600 such that the manipulation system 1003 is positioned adjacent the implant (e.g., prosthetic valve 1001) associated with the delivery device 1500 of the delivery system 1002.

As indicated in FIGS. 1A-1D, the manipulation device 1400 may include an elongate portion 1401 (e.g., a filament, wire, or the like) that terminates at and is coupled to an engaging portion 1402 at or near a distal end of the manipulation device 1400. For reference, the engaging portion 1402 is indicated generally as a generic box and may take a variety of forms including any of those subsequently described. Generally, the engaging portion 1402 may have any suitable shape, size, and overall configuration that facilitates manipulation of an internal body structure by manipulating the elongate portion 1401. As shown, the constraining sheath 1600 that receives the manipulation device 1400 includes an elongate tubular construct that slidably receives the manipulation device 1400, or at least a portion thereof.

In some examples, the constraining sheath 1600 is constrained by or otherwise coupled to the implant (e.g., prosthetic valve 1001) (the prosthetic valve 1001 as shown in FIGS. 1C and 1D). In particular, in FIG. 1C, the constraining sheath 1600 is coupled with the prosthetic valve 1001 via the anchor frame subcomponent 1100 by passing through an open framework of a support structure (e.g., stent structure) of the anchor frame subcomponent 1100 and/or by penetrating through a film or fabric associated with the support structure of the anchor frame subcomponent 1100. In some examples, the constraining sheath 1600 is coupled with the anchor frame subcomponent 1100 by passing through a closed cell aperture or void formed in the framework of the anchor frame subcomponent 1100. In still other examples, the constraining sheath 1600 is coupled with the leaflet frame subcomponent 1200. In FIG. 1D, according to some examples, the constraining sheath 1600 is coupled with the prosthetic valve 1001 via the interstage 1300. The interstage 1300 includes an open framework that is optionally covered with a film or fabric defining a conduit 1302. As shown, the constraining sheath 1600 may pass through an opening in the conduit 1302. Where the interstage 1300 includes an open framework and no cover, the constraining sheath 1600 may pass through the open framework such that the constraining sheath 1600 is coupled to the interstage 1300.

FIG. 1E is a schematic view of a longitudinal section of a portion of the constraining sheath 1600 and manipulation device 1400, which shows a general inter-relation of components, according to some examples. Though the engaging portion 1402 may be formed as an integral component with the elongate portion 1401, in some examples the engaging portion 1402 is a separate component coupled (e.g., releasably coupled) to the elongate portion 1401. As shown schematically, the engaging portion 1402 is positioned at or near a distal end of the elongate portion 1401.

As in FIGS. 1A-1D, the engaging portion 1402 is also shown as a generic box in FIG. 1E to reflect that the engaging portion 1402 may take a variety of forms and shape, including any of those described herein. In some examples, the engaging portion 1402 may be a self-expanding anchor with one or more hooks, prongs, projections, screws, or other anchoring features. The engaging portion 1402 may have a relative sharp tip, or alternatively, an atraumatic tip as desired. The elongate portion 1401 may be configured as a wire, filament, tube, rod, or any other suitable configuration.

In the example shown schematically in FIG. 1E, the elongate portion 1401 and the engaging portion 1402 are coupled with each other via a connection member 1404 which, in some embodiments, releasably attaches the engaging portion 1402 to the manipulation device 1400. In some examples, the connection member 1404 is a wire, string, thread, clip, or any other suitable means for coupling, including releasably coupling, the engaging portion 1402 of the manipulation device 1400 to the elongate portion 1041. In some embodiments, the engaging portion 1402 may be detached or uncoupled from the manipulation device 1400, for example, by applying a predetermined amount of force to the manipulation device 1400 in the proximal direction.

FIG. 1F is another generalized representation showing additional features of the transcatheter system 1000, according to some embodiments. As indicated generally in FIG. 1F, components of the transcatheter system 1000, such as the delivery system 1002 and the manipulation system 1003 are controlled by a handle assembly 2000 at a body proximal end 2001 of the transcatheter system 1000, in accordance with an embodiment. Each of the delivery system 1002 and the manipulation system 1003 are operable to extend from a body distal end 2002 of the transcatheter system body, such as the delivery sheath 1504.

As previously described, the delivery device 1500 of the delivery system 1002 and the constraining sheath 1600 are positioned within the delivery sheath 1504. The delivery sheath 1504, as well as the delivery device 1500 and the constraining sheath 1600, may be operatively arranged relative to one another (e.g., longitudinally slid or axially rotated relative to one another) using one or more handles in the handle assembly 2000 positioned at the body proximal end 2001 of the manipulation system 1003. In some examples, the handle assembly 2000 is capable of independently adjusting the positions of the delivery system 1002 (e.g. delivery device 1500 and delivery sheath 1504) and a manipulation system 1003 (e.g. manipulation device 1400 and constraining sheath 1600) before, during, and/or after deployment. For example, the handle assembly 2000 may be capable of proximally retracting the delivery sheath 1504 relative to the delivery device 1500 while maintaining the position of the delivery device 1500 to deploy the implant (e.g., reveal or release the prosthetic valve 1001, allowing the prosthetic valve 1001 to assume the expanded configuration). The handle assembly 2000 may then proximally retract the constraining sheath 1600 to deploy the manipulation device 1400. Additionally, the handle assembly 2000 may also be capable of distally extending the delivery sheath 1504 or the constraining sheath 1600 as desired.

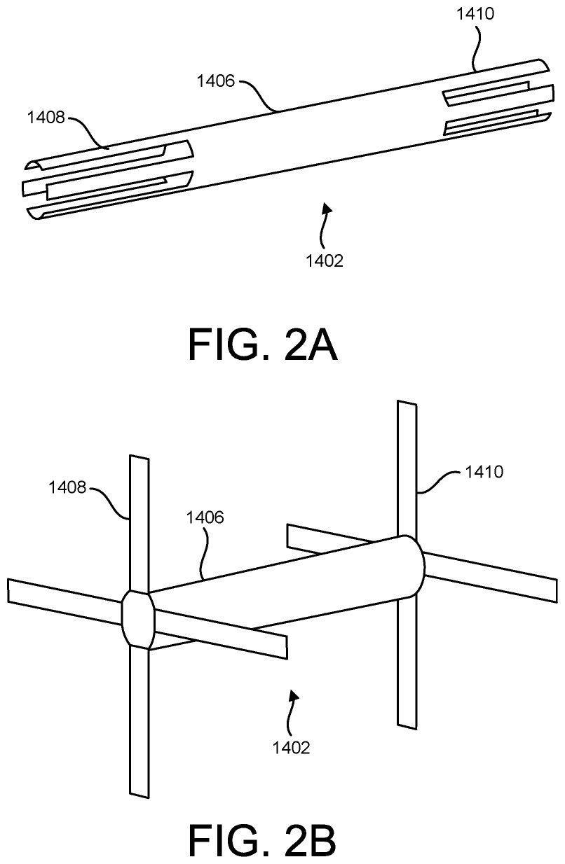

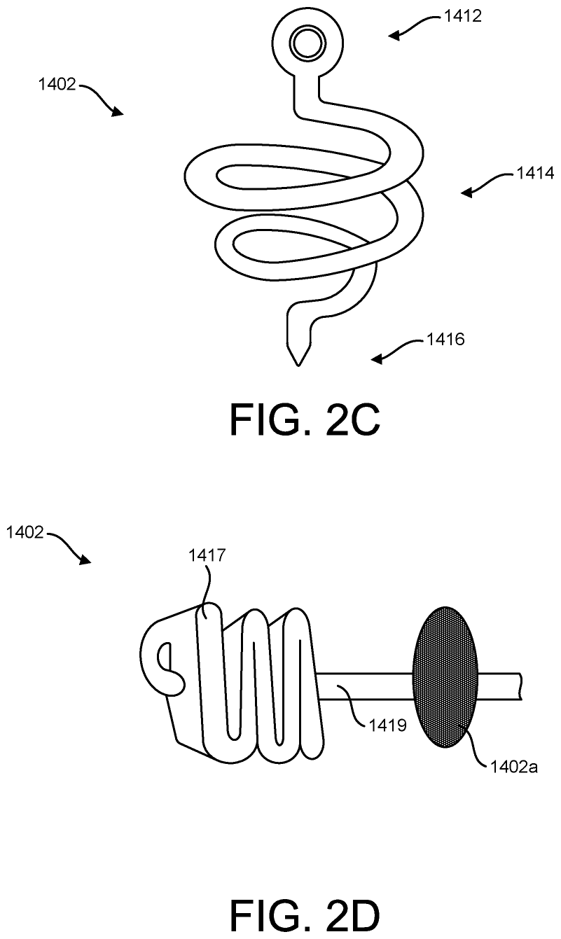

FIGS. 2A through 2C show some non-limiting examples of the engaging portion 1402 configured as an anchor component. In FIG. 2A, an example of a “spider” configuration for the engaging portion 1402 is shown in a compressed or restricted configuration while inside the constraining sheath 1600. The engaging portion 1402 in this example has an elongated body that includes a main body portion 1406, a proximal leg portion 1408, and a distal leg portion 1410. The proximal leg portion 1408 and the distal leg portion 1410 are formed via a plurality of cuts, reliefs, or gaps along a portion of the length of the engaging portion 1402 such that, when the engaging portion 1402 is deployed as shown in FIG. 2B, the proximal leg portion 1408, including a plurality of legs, and the distal leg portion 1410, including a plurality of legs, deploy (e.g., radially expand such that the pluralities of legs of each expand outward away from each other). In at least this manner, the proximal leg portion 1408 and distal leg portion 1410 function as anchors when placed against tissue surface(s), since the radial extension of the engaging portion 1402 is greater at each of the proximal leg portion 1408 and the distal leg portion 1410 than in the main body portion 1406 disposed therebetween. In some alternative examples, the engaging portion 1402 only includes a plurality of legs at one end (e.g., at the proximal leg portion 1408 or the distal leg portion 1410), but not both. Though separate, coupled components are described, in some examples, the engaging portion 1402 (e.g., the proximal leg portion 1408 thereof) may be continuous with the elongate portion 1401 of the manipulation device 1400.

FIG. 2C shows an example of a “pigtail,” helical, or corkscrew configuration for the engaging portion 1402 in an expanded configuration. This engaging portion 1402 may be in a straight or essentially straight configuration while inside the constraining sheath 1600 in a pre-deployed configuration. When in a deployed configuration (e.g., extended from the constraining sheath 1600), a portion of the engaging portion 1402 radially expands and forms a shape resembling a helical spiral (or “pigtail”). As shown, the expanded configuration of the engaging portion 1402 has a coupling section 1412, an anchoring section 1414, and a piercing section 1416, where the anchoring section 1414 is disposed between the coupling section 1412 and the piercing section 1416.

In some examples, the coupling section 1412 functions as part of a coupling mechanism that facilitates detachably coupling the engaging portion 1402 with the manipulation device 1400 (e.g., via the connection member 1404). In some examples, the connection member 1404 is looped through the coupling section 1412 such that the connection member 1404 is releasable therefrom (e.g., by breaking the connection member 1404, the coupling section 1412, or via another release mechanism). The coupling section 1412 may include an aperture, hook, notch, or any other suitable configuration that assists with permanently or releasably coupling to the connection member 1404 or directly to the elongate portion 1401 of the manipulation device 1400.

The piercing section 1416 is configured to pierce or puncture tissue surfaces to assist with coupling the engaging portion 1402 with tissue to be manipulated. The piercing section 1416 may be relatively straight (e.g., to directly pierce a surface) or curved (e.g., to hook into a surface) as desired. In some deployment examples, the piercing section 1416 is the first section of the engaging portion 1402 that is deployed from the constraining sheath 1600. If desired, the piercing section 1416 may be distally advanced from the constraining sheath 1600 with the constraining sheath adjacent or against the tissue to be manipulated to help the piercing section 1416 pierce the tissue surface. Remaining portions of the engaging portion 1402 may then be advanced through the tissue as desired.

In various examples, the anchoring section 1414 helps hold, grasp, retain, stow, capture, seize, or otherwise anchor to a portion of a tissue such that the tissue is coupled, secured, or otherwise anchored to the engaging portion 1402. In some methods of use, the anchoring section 1414 has a spiral structure and engages the tissue after the piercing section 1416 pierces through the tissue, and the operator rotates the engaging portion 1402. In some examples, the engaging portion 1402 is rotated by rotating the elongate portion 1401 using the handle assembly 2000. For example, rotating the anchoring section 1414 having a spiral configuration in a first direction may cause the engaging portion 1402 to advance into the pierced tissue until at least a portion of the tissue is engaged and retained by the anchoring section 1414. The tissue may then be manipulated (e.g., moved distally or proximally) by actuating the manipulation device 1400 (e.g., by retracting or extending the elongate portion 1401 using the handle assembly 2000).

FIG. 2D shows another engaging portion 1402 configuration in the form of pledget anchor design. In particular, the engaging portion 1402 in FIG. 2D includes a pledget anchor 1417 and a tether portion 1419 with an associated stop mechanism 1402a. The pledget anchor 1417 may be expanded to an anchoring configuration by tensioning the tether portion 1419. The stop mechanism 1402a may be an elastomeric member configured to slide over the tether portion 1419 with sufficient tension but to retain its position after “cinching” or otherwise sliding the stop mechanism 1402a along the tether portion 1419 to define a set distance between the pledget anchor 1417 and the stop mechanism 1402a. In this manner, tissue (e.g., the anterior leaflet 12) may be “cinched” or compressed between the pledget anchor 1417 and the stop mechanism 1402a.

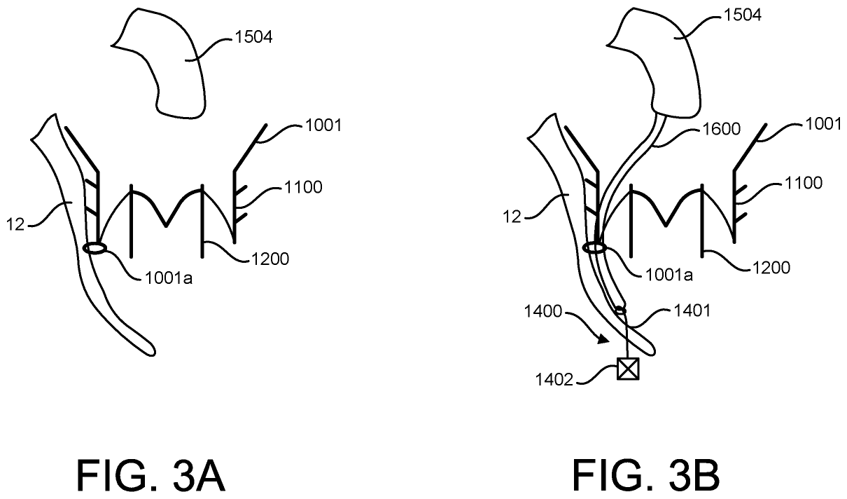

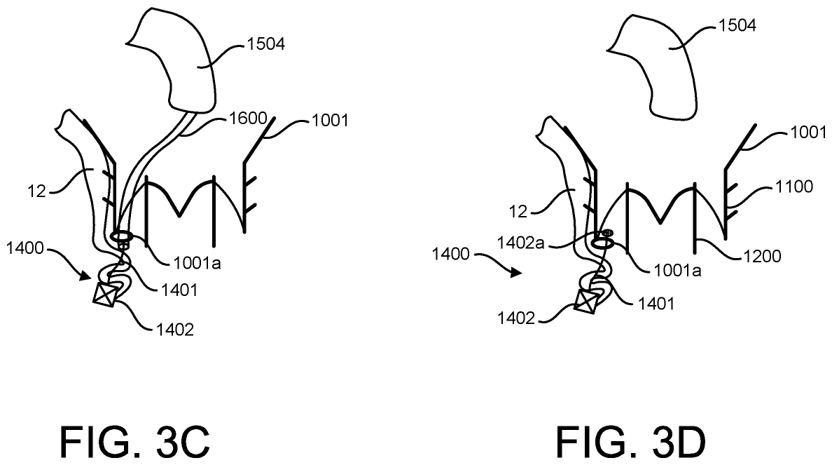

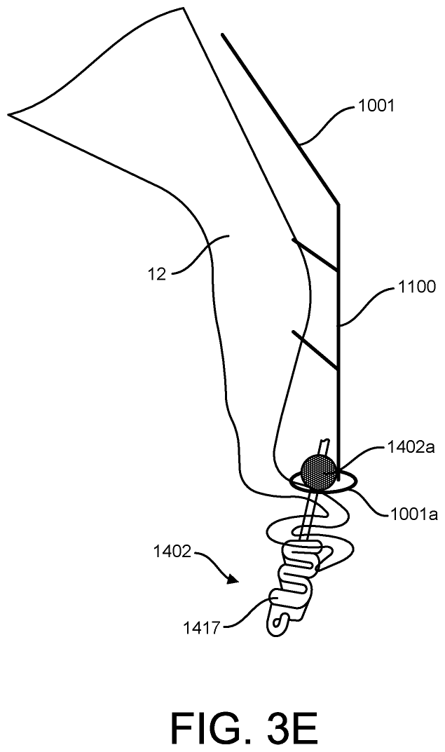

FIGS. 3A to 3D illustrate a leaflet manipulation operation using the manipulation device 1400, according to various examples. As shown in FIG. 3A, according to some examples the prosthetic valve 1001 is implanted in a native valve orifice, such as a native mitral valve orifice. As shown, a portion of the anterior leaflet 12 extends beyond the prosthetic valve 1001. In such an instance, the anterior leaflet 12 may impede proper functioning of the prosthetic valve 1001, including potentially blocking the left ventricular outflow tract (LVOT).

As shown in FIG. 3B, the manipulation device 1400 of the manipulation system 1003 is extended through the constraining sheath 1600 toward a portion of the anterior leaflet 12 (e.g., near the free edge of the anterior leaflet 12). The manipulation device 1400 has been extended to pierce the anterior leaflet 12, with the engaging portion 1402 having passed through the anterior leaflet 12 to an opposite side of the anterior leaflet 12. If desired, prosthetic valve 1001 optionally includes a guide feature 1001a (e.g., a ring or hoop of material) that is coupled to a portion of the prosthetic valve, such as the anchor frame subcomponent 1100. The guide feature 1001a may be used to assist with positioning the manipulation device 1400 in the desired location.

As shown in FIG. 3C the manipulation device 1400 is retracted to bunch, compress, or otherwise manipulation a portion of the anterior leaflet 12. As shown, the anterior leaflet 12 may be retracted to help prevent LVOT obstruction as desired.

As shown in FIG. 3D, the manipulation device 1400 is optionally used to push stop mechanism 1402a to a desired position to retain the leaflet tissue in the desired, retracted position. In some examples, the stop mechanism 1402a engages with the guide feature 1001a so that the engaging portion 1402 is tethered or otherwise coupled to the prosthetic valve 1001, and thus the position of the anterior leaflet 12 is maintained. In some examples, the engaging portion 1402 is freed from the remainder of the manipulation device 1400 (e.g., the engaging portion 1402 is released from the elongate portion 1401) and the manipulation system 1003 is retracted or otherwise removed from the treatment site.

FIG. 3E is an enlarged view of an example where the engaging portion 1402 is configured as a pledget anchor, as described in association with FIG. 2D. As shown, the anterior leaflet 12 is gathered with the pledget anchor 1417 on one side of the tissue and the stop mechanism 1402a on an opposite side of the tissue to retain the anterior leaflet 12 in the retracted position shown. As shown generally, the stop mechanism 1402a is retained by the guide feature 1001a associated with the prosthetic valve 1001 (only one side of the prosthetic valve 1001 is shown in FIG. 3E).

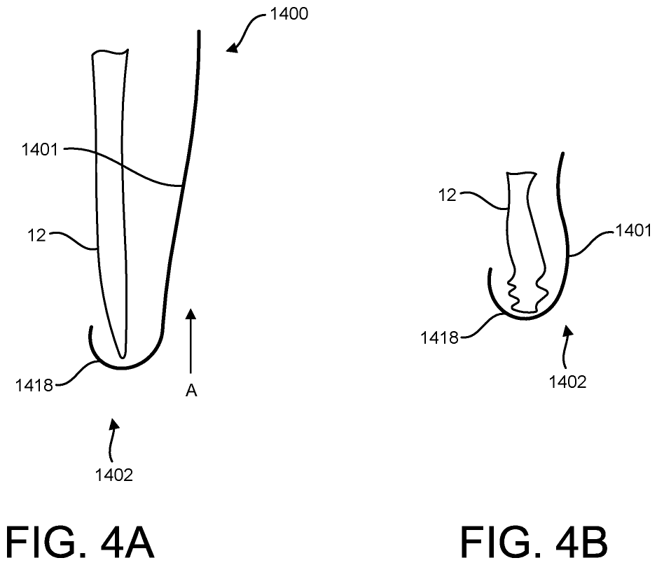

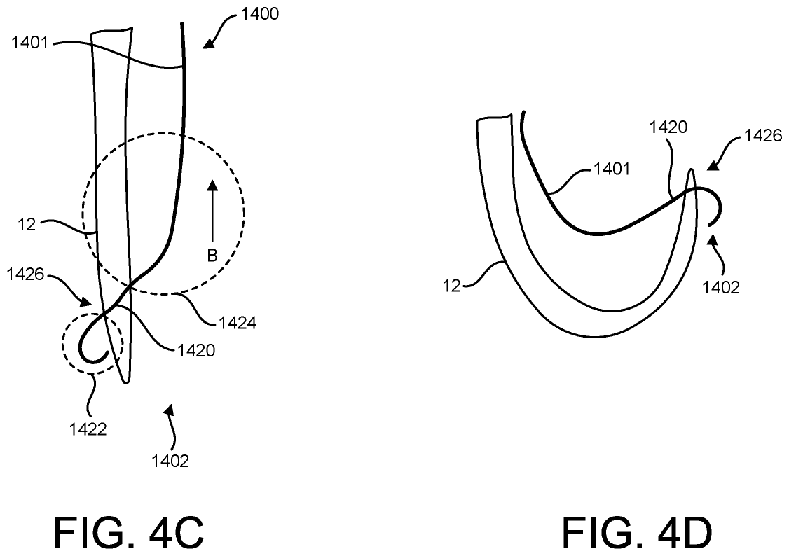

FIGS. 4A, 4B, 4C, and 4D show examples of the manipulation device 1400 with additional configurations for the engaging portion 1402. As shown in those examples, the elongate portion 1401 and the engaging portion 1402 may be integrally formed as a single, continuous unit. For example, the manipulation device 1400 may be formed as a wire (e.g., monofilament, multifilament, tubular, or other configuration), with the engaging portion 1402 including one or more curved sections, or curves, such as a distal curved section 1418. As shown in FIGS. 4A and 4B, the distal curved section 1418 defines a hook shape such that the engaging portion 1402 can be described as a leaflet hooking feature. The distal curved section 1418 may be configured to curve around and receive a tissue structure, such as an anterior leaflet 12 of a heart valve (e.g., the mitral valve). The engaging portion 1402 may be pulled or retracted in the direction of an arrow A as shown in FIG. 4A to pull or retract the received tissue structure, which is the anterior leaflet 12 as shown in FIG. 4A. By way of example only, the radius of curvature for the distal curved section 1418 may be from about 1 mm and to about 2 mm, from about 2 mm and to about 3 mm, from about 3 mm and to about 4 mm, greater than 4 mm, or any range therebetween.

In some examples, pulling or retracting the engaging portion 1402 (e.g., by pulling or retracting the elongate portion 1401, such as by actuating the handle assembly 2000) causes the received tissue structure (e.g., anterior leaflet 12) to be captured and retracted in the direction in which it is pulled (e.g., as shown in FIG. 4B). In some examples, the anterior leaflet 12 (or other tissue structure) is retracted using the manipulation device 1400 for receipt between implant (e.g., prosthetic valve 1001) and surrounding tissue (e.g., the native mitral valve orifice). Thus, some methods of implanting the prosthetic valve 1001 include retracting the anterior leaflet 12 for positioning between the prosthetic valve 1001 and the native mitral valve orifice. In some examples, the anterior leaflet 12 is retracted into a space between the prosthetic valve 1001 and surrounding tissue following valve expansion and deployment, and in others the anterior leaflet 12 is retracted prior to valve expansion and deployment.

In some examples, rather that hooking or receiving tissue structures, the engaging portion 1402 is additionally or alternatively configured to pierce the tissue structure. FIGS. 4C and 4D illustrate an example of such a piercing configuration. As shown, the engaging portion 1402 includes two or more curvatures in different or opposing directions proximate to the distal end to collectively define a recurved hook 1420. In some examples, the recurved hook 1420 has a first curved section 1422 with a smaller radius of curvature at or proximate to the distal end, and a second curved section 1424 that has a different curvature (e.g., in an opposition direction and/or with a different radius of curvature) than the first curved section 1422. The second curved section 1424 may have a larger radius of curvature than the first curved section 1422, and the second curved section 1424 may be located farther away from the distal end than the first curved section 1422. In some examples, the first curved section 1422 and the second curved section 1424 are curved in different directions (e.g., opposite directions) from one another. For example, the first curved section 1422 may have a clockwise curvature and the second curved section 1424 may have a counterclockwise curvature when viewed from the same angle.

In some examples where the internal body structure, or tissue structure to be manipulated is the anterior leaflet 12, the recurved hook 1420 is configured to facilitate puncturing of the anterior leaflet 12 at a location 1426 (e.g., toward a free edge of the anterior leaflet 12). For example, the first curved section 1422 is optionally configured to pierce and anchor to the anterior leaflet 12. The manipulation device 1400 may be pulled or retracted in the direction indicated by an arrow B in FIG. 4C (e.g., using the handle assembly 2000 as previously described). In some examples, the radius of curvature for the first curved section 1422 may be from about 1 mm and to about 2 mm, from about 2 mm and to about 3 mm, from about 3 mm and to about 4 mm, greater than 4 mm, or any range therebetween, although a variety of values are contemplated. In some examples, the radius of curvature of the second curved section 1424 is greater than that of the first curved section 1422 such that the second curved section 1424 forms a larger, or less abrupt curve than the first curved section 1422. In some examples, the second curved section 1424 is configured to help position the first curved section 1422 at a desired position relative to the tissue structure (e.g., the anterior leaflet 12) to be manipulated. As in prior examples, pulling or retracting the engaging portion 1402 (e.g., by pulling or retracting the elongate portion 1401, such as by actuating the handle assembly 2000) causes the internal bodily structure (e.g., anterior leaflet 12) to be retracted in the direction in which it is pulled (e.g., as shown in FIG. 4D) to take on a more folded, compacted, retracted or otherwise gathered configuration.

In some examples, the anterior leaflet 12 (or other tissue structure) is retracted using the manipulation device 1400 of FIGS. 4C and 4D for receipt between implant (e.g., prosthetic valve 1001) and surrounding tissue (e.g., the native mitral valve orifice). Thus, some methods of implanting the prosthetic valve 1001 include retracting the anterior leaflet 12 for positioning between the prosthetic valve 1001 and the native mitral valve orifice.

In some examples, the anterior leaflet 12 is retracted into a space between the prosthetic valve 1001 and surrounding tissue following valve expansion and deployment, and in others the anterior leaflet 12 is retracted prior to valve expansion and deployment. The manipulation device 1400 examples of FIGS. 4A to 4D may be configured similarly to a guidewire as desired, and may be sufficiently thin or narrow to be slidable from the space between the valve orifice and the prosthetic valve 1001 after the anterior leaflet 12 has been retracted and the implant (e.g., prosthetic valve 1001) is expanded. In some examples, the manipulation device 1400 straightens when sufficient tension is applied, where the manipulation device 1400 elastically deforms at the first curved section 1422 and/or the second curved section 1424 to release the anterior leaflet 12 (or other internal bodily structure) and facilitate retraction of the manipulation device 1400 from the treatment site.

FIGS. 5A to 5D illustrate a non-limiting example of a deployment sequence and retrieval operation using the manipulation system 1003 during prosthetic valve 1001 implantation (e.g., prosthetic mitral valve). FIG. 5A shows a manipulation system 1003 at a treatment site in a body of a patient according to some embodiments. The manipulation system 1003 is inserted into a left atrium 22 such that a catheter 102 of the manipulation system 1003 resides in the left atrium 22 while a manipulation device 1400 extends from the delivery sheath 1504 (e.g., a catheter) and passes through a valve orifice 26, such as a mitral valve, which includes the anterior leaflet 12 and a posterior leaflet 24, and into a left ventricle 18. The distal curved section 1418 of the engaging portion 1402 (for example those as shown in FIGS. 1A through 1D) of the manipulation device 1400 is in the shape of a hook, in accordance with an embodiment. The left ventricle 18 includes a plurality of papillary muscles 16 from which chordae tendineae 14 extend to the anterior leaflet 12 or the posterior leaflet 24.

FIG. 5B shows the manipulation device 1400 in an engagement position following the position shown in FIG. 5A. In this engagement position, the engaging portion 1402 engages the anterior leaflet 12, such as by grasping an edge of the anterior leaflet 12, and retracting the manipulation device 1400 such that the anterior leaflet 12 is positioned to protrude less in the left ventricular outflow tract (“LVOT”), which is drawn adjacent to the tissue annulus in the figures, thus minimizing the amount of obstruction caused by the anterior leaflet 12 when a prosthetic valve 1001 is inserted into the valve orifice 26 between the anterior leaflet 12 and the posterior leaflet 24, as seen in FIG. 5C.

FIG. 5C shows the prosthetic valve 1001 positioned at the valve orifice, with the anterior leaflet 12 manipulated such that it resides between the valve orifice 26 and the prosthetic valve 1001, and is held in position by the engaging portion 1402 of the manipulation device 1400. For example, the prosthetic valve 1001 may be delivered through the delivery sheath 1504 in a radially compressed form. In various examples, the prosthetic valve 1001 is coupled to the delivery device 1500 inside the delivery sheath 1504, and the prosthetic valve 1001 is extended into the left ventricle 18 after the anterior leaflet 12 is retracted by the distal curved section 1418, such as the hook. The manipulation device 1400 may remain positioned between the valve orifice 26 and the prosthetic valve 1001 in this configuration. After the prosthetic valve 1001 is positioned at the valve orifice 26, the prosthetic valve 1001 may be radially expanded from a compacted configuration to an expanded configuration to engage the valve orifice 26. FIG. 5D shows the prosthetic valve 1001 expanded to a deployed, or expanded configuration with the manipulation device 1400 and the delivery device 1500 of the delivery system 1002 retracted from the treatment site, leaving the prosthetic valve 1001 behind. As previously indicated, the prosthetic valve 1001 may be self-expanding with the delivery system 1002 configured to permit self-expansion of the prosthetic valve 1001 at a desired time or radially expandable using an expansion member, such as an expandable balloon associated with a balloon catheter.

In some examples, the engaging portion 1402 of the manipulation device 1400 is retracted and retrieved by extending the constraining sheath 1600 distally along the elongate portion 1401 of the manipulation device 1400 such that the constraining sheath 1600 straightens, or diametrically compacts the engaging portion 1402 of the manipulation device 1400. In this manner, the constraining sheath 1600 may be utilized to facilitate straightening or other diametric compaction of the engaging portion 1402 to facilitate removal from the treatment site.

A variety of materials may be utilized to form the elongate portion 1401 and the engaging portion 1402 of the manipulation device 1400. For example, the manipulation device 1400 may comprise, such as, but not limited to, any elastically deformable metallic or polymeric biocompatible material, in accordance with embodiments. The manipulation device 1400 may comprise a shape-memory material, such as nitinol, a nickel-titanium alloy. Other materials suitable for the manipulation device 1400 include, but are not limited to, other titanium alloys, stainless steel, cobalt-nickel alloy, polypropylene, acetyl homopolymer, acetyl copolymer, other alloys or polymers, or any other biocompatible material having adequate physical and mechanical properties to function as a manipulation device as described herein.

In accordance with some embodiments, the manipulation device 1400 comprises an elastic material (e.g., a shape memory material) and is operable to flex under load and retain its original shape when the load is removed, thus allowing the manipulation device 1400 to self-expand from a compressed shape (e.g., elongated or relatively more linear shape) to a predetermined shape (e.g., expanded or relatively more curved or angular shape). In different terms, the manipulation device 1400 may be elastically deformable to be self-expanding when freed from external constraint (e.g., when extended from the constraining sheath 1600 and compactible or collapsible to a straighter or diametrically compact configuration when placed under external constraint (e.g., when positioned within or retracted into the constraining sheath 1600). In some other examples, the manipulation device 1400 includes one or more portions configured to be plastically deformed and one or more portions configured to be elastically deformed. That is, in some examples, the manipulation device 1400 includes one or more elastically deformable components or features and one or more plastically deformable components or features. Thus, it should be appreciated that the examples of the manipulation device 1400 presented herein are not to be limited to a specific design or mode of expansion or deformation.

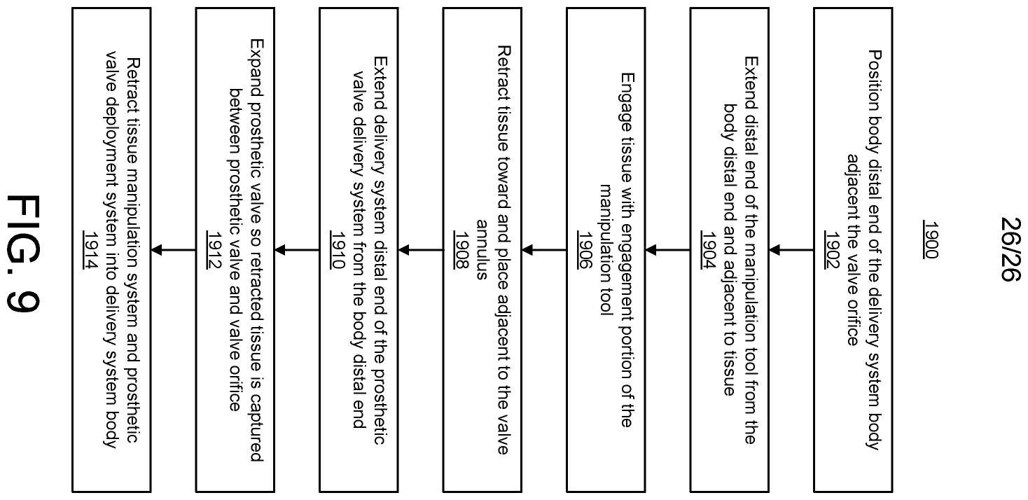

FIG. 9 shows a process flow fora method 1900 of delivering and deploying a prosthetic valve into a valve orifice, and associated tissue manipulation for manipulating tissue and capturing it between the prosthetic valve and the native valve orifice using the transcatheter system according to any of the examples provided herein. The method may include manipulating and capturing an anterior leaflet of a mitral valve to avoid left ventricular outflow tract obstruction. The distal end of the delivery sheath of the transcatheter system may be positioned adjacent the valve orifice; 1902. The distal end of the manipulation device may be extended and positioned adjacent tissue to be manipulated; 1904. In some examples, the tissue may be that of the anterior leaflet. The tissue may be engaged by the engaging portion 1402 of the manipulation device; 1906.

In some examples, the tissue is engaged by using the engaging portion 1402 of the manipulation device to hold, grasp, retain, capture, or seize a portion of the tissue. In some examples, the engaging portion 1402 punctures the tissue. The tissue may be retracted toward and placed adjacent to the valve annulus using a manipulation device; 1908. The prosthetic valve may be positioned and expanded within the valve orifice with the retracted tissue between the prosthetic valve and the valve orifice; 1910. For example, the prosthetic valve may be expanded such that the retracted tissue is captured between the prosthetic valve and the valve orifice; 1912. In some examples, the prosthetic valve is first expanded, where step 1912 occurs prior to retracting tissue, and then the manipulation device is utilized to retract tissue. For example, as described in association with FIGS. 3A to 3D.

Regardless, in some methods, following tissue manipulation, the manipulation system and the prosthetic valve delivery system are retracted into the delivery sheath; 1914. In some examples, the manipulation system may be retracted by first extending the constraining sheath 1600 along the manipulation device 1400 to straighten the engaging portion 1402 and then retracting the constraining sheath 1600. In other examples, the manipulation device 1400 (e.g., the elongate portion 1401 and/or the engaging portion 1402) is additionally or alternatively straightened by exerting enough strain on the curved portion(s) of the manipulation system to cause them to straighten. In some examples, retraction of the manipulation device 1400 may include releasing or uncoupling the engaging portion 1402 from the elongate portion 1401. The disclosed methods are not to be understood to be strictly sequential, and therefore may be implemented in a different order than discussed and where appropriate.

### Prosthetic Valve Delivery System Examples

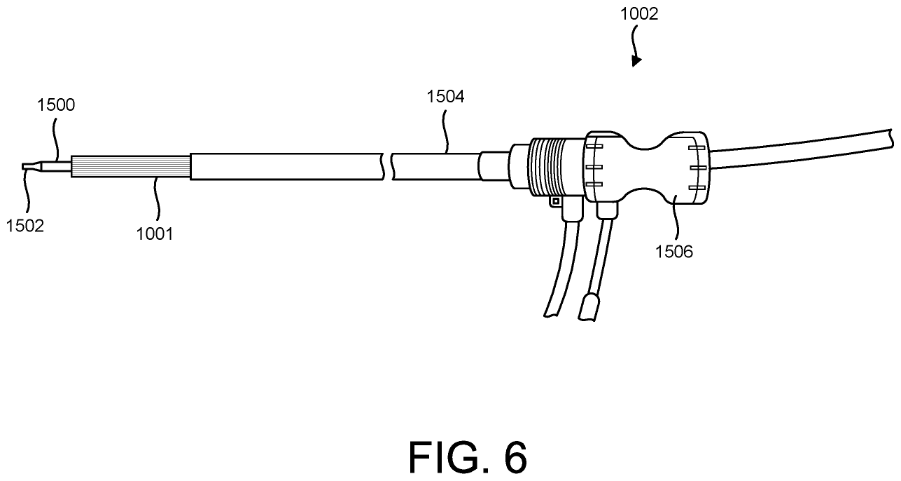

FIG. 6 shows an example of the delivery system 1002 configured for use with prosthetic valve 1001, including the delivery sheath 1504, the delivery device 1500 such as a delivery catheter, and the prosthetic valve 1001 maintained in a collapsed configuration within the delivery sheath 1504 proximate to a tip portion 1502 of the delivery device 1500. As shown, the prosthetic valve 1001 is arranged on the delivery device 1500 in a distally extended position from the delivery sheath 1504. Or, in different terms, the prosthetic valve 1001 is in an extended position. The prosthetic valve 1001 can be substituted with a variety of implants, including self-expanding implants, such as a stent, stent graft, occluder, or vascular filter, for example. As shown, the delivery sheath 1504 may be an introducer sheath including a hemostatic valve 1506, for example, although any of a variety of additional or alternative features are contemplated. Self-expanding, radially expandable (e.g., balloon expanding), and combinations of self-expanding and radially expandable delivery system designs are contem plated.

### Prosthetic Valve Examples

The following provides additional implant examples and features (in the context of the prosthetic valve 1001), useable with the delivery system 1002, and the manipulation system 1003. Though examples are provided, it is to be understood additional one-way valve designs are known and may be used in the transcatheter system 1000.

FIGS. 7A and 7B show a first example of the prosthetic valve 1001, which includes a leaflet frame subcomponent 1200 that helps provide the prosthetic valve 1001 with the functionality of a one-way valve. It is also appreciated that, for transcatheter applications, the leaflet frame subcomponent 1200 is has a smaller-diameter compressed configuration and a larger-diameter expanded configuration, and that the one-way valve component should accommodate that functionality. The leaflet frame subcomponent 1200 can be configured to be received within at least a portion of an anchor frame subcomponent 1100.

### Leaflet Frame Subcomponent

The leaflet frame subcomponent 1200 provides the prosthetic valve 1001 with the functionality of a one-way valve. It is understood and appreciated that one-way valves are well known in the art and may be used herein. It is appreciated that mechanical valves, biological valves, and biological and synthetic leaflet valves may be used as the one-way valve of the leaflet frame subcomponent 1200. It is also appreciated that, for transcatheter applications, the leaflet frame subcomponent 1200 includes a smaller-diameter compressed configuration and a larger-diameter expanded configuration, and that the one-way valve component is able to accommodate that functionality.

The leaflet frame subcomponent 1200 may include a leaflet frame 1201 and an optional cover material (not shown for ease of visualization of the leaflet frame 1201). In different terms, the side of the leaflet frame 1201 may be at least partially covered, such as with a film or fabric (not shown for clarity) suitable for a particular purpose, such as, but not limited to, to restrict fluid from passing through apertures of the leaflet frame 1201. For illustrative purposes, the following examples are suitable especially for a transcatheter application, but are also suitable for a surgical application. The leaflet frame subcomponent 1200 includes a leaflet frame 1201 and leaflets (e.g., leaflets 1814, FIG. 8E).

In various examples, the leaflet frame 1201 (and anchor frame 1101) is elastically deformable so as to be self-expanding under spring bias forces, as those of skill will appreciate. In some examples, the leaflet frame 1201 is plastically deformable so as to be mechanically expanded such as with a balloon, as those of skill will appreciate. In yet some other examples, the leaflet frame 1201 is plastically deformable as well as elastically deformable. That is, in some examples, the leaflet frame 1201 includes one or more elastically deformable components or features and one or more plastically deformable components or features. Thus, it should be appreciated that the examples of the leaflet frame 1201 presented herein are not to be limited to a specific design or mode of expansion.

The leaflet frame 1201 (and the anchor frame 1101) can be made of any of various suitable plastically-expandable materials (e.g., stainless steel, etc.) or self-expanding materials (e.g., nickel titanium alloy (NiTi), such as nitinol), in accordance with embodiments. When constructed of a plastically-expandable material, the leaflet frame 1201, and thus the prosthetic valve 1001, can be compressed to a radially collapsed configuration in the delivery device 1500 and then expanded inside a patient by an inflatable balloon or equivalent expansion mechanism (not shown). When constructed of a self-expandable material, the leaflet frame (1201), and thus the prosthetic valve 1001, can be crimped to a radially collapsed configuration and restrained in the collapsed configuration by insertion into a sheath or equivalent mechanism of the delivery device 1500. Once inside the body, the prosthetic valve 1001 can be advanced from the delivery sheath which allows the prosthetic valve to expand to its functional size.

Suitable plastically-expandable materials that can be used to form the leaflet frame 1201 include, without limitation, stainless steel, biocompatible alloys (e.g., cobalt-chromium or nickel-cobalt-chromium alloys), polymers, or combinations thereof. In some embodiments, the frame is made of a nickel-cobalt-chromium-molybdenum alloys, such as MP35N® alloy (SPS Technologies, Jenkintown, Pa.).

In some methods of making the leaflet frame 1201, a pattern of the apertures or cells may be cut into a tube to form an annular-shaped cut stent frame or support structure defining the leaflet frame 1201. As shown, the cells may generally be arranged into rows forming the annular shape of the support structure. The leaflet frame 1201 may be formed or otherwise configured such that the cells have differing sizes and/or configurations (e.g., one or more rows of smaller cells and one or more rows of larger cells as shown). It is appreciated that the leaflet frame 1201 can be formed via various manufacturing processes, including, but not limited to, additive manufacturing, subtractive manufacturing, and injection molding. For example the frame may be formed of wire or braided wire, formed by three-dimensional printing, as well as formed by etching, cutting, laser cutting, and stamping sheets or tubes of material, among other suitable processes, into an annular or tubular structure or, if a sheet of material, with the sheet then formed into an annular or tubular structure. In various examples, the frame can comprise, such as, but not limited to, any biocompatible and, in those embodiments where applicable, elastically deformable metallic or polymeric material including shape-memory materials, such as nitinol, a nickel-titanium alloy. Other materials suitable for the frame include, but are not limited to, other titanium alloys, stainless steel, biocompatible alloys (e.g., cobalt-chromium alloy, cobalt-nickel alloy, nickel-cobalt-chromium alloy, or nickel-cobalt-chromium-molybdenum alloy, such as MP35N® alloy (SPS Technologies, Jenkintown, Pa.)), polymers, polypropylene, polyethylene terephthalate, PEEK, acetyl homopolymer, acetyl copolymer, other alloys, polymers, and thermoplastics, or any other material that is generally biocompatible having adequate physical and mechanical properties to function as a frame as described herein, or combinations thereof.

It is understood that when the frame is constructed of a plastically-deformable material, the frame, and thus the prosthetic valve, can have a lower radial dimension when coupled to the delivery catheter and then can be expanded to a larger radial dimension inside a patient, either by internal forces or by external forces, such as by an inflatable balloon or equivalent expansion mechanism. When constructed of an elastic material, the frame, and thus the prosthetic valve, can be crimped to a radially collapsed configuration and restrained in the collapsed configuration by, for example, insertion into a sheath or constrained by fibers or other constraining mechanism. Once inside the body, the prosthetic valve can be released from the constraining mechanism, which allows the prosthetic valve to expand to its functional size.

In various embodiments, the leaflet frame subcomponent 1200 additionally supports or otherwise includes a valve structure. In some examples, the valve structure includes one or more leaflets (e.g., leaflets 1814, FIG. 8E). A variety of mechanical valve leaflet, biological leaflet, and synthetic leaflet designs are known in the medical technology arts, any of which may be incorporated into the leaflet frame subcomponent 1200 of the present disclosure.

In some examples, the leaflets are coupled to the leaflet frame subcomponent interior surface (e.g., to the leaflet frame interior surface). The leaflets may be coupled to the leaflet frame subcomponent 1200 in any of a variety of manners, including by wrapping one or more portions of the leaflets about one or more portions of the leaflet frame 1201, by suturing or sewing one or more portions to the leaflet frame 1201, but using adhesives or other mechanical fasteners, or by using one or more projections on the leaflet frame 1201 (not shown) that pass through one more apertures configured to be disposed about the one or more projections (not shown). Any of these coupling mechanisms, combinations thereof, and others may be employed as appropriate.

The leaflet frame subcomponent 1200 is configured to be received within at least a portion of the anchor frame subcomponent 1100, as will be described in more detail below. It will be appreciated that nonlimiting examples of leaflet frame subcomponent 1200 can be provided with a diameter (e.g., a diameter of an interior or exterior surface of the leaflet frame subcomponent 1200) in a range from about twenty (20) millimeters and to about thirty (30) millimeters, depending on a patient's anatomy, although a variety of dimensions are contemplated.

It will be appreciated that in some embodiments, the leaflet frame subcomponent 1200 may be operable to engage directly with the patient anatomy (e.g., native mitral valve orifice). According to some embodiments, the prosthetic valve 1001 may include a leaflet frame subcomponent 1200 and does not require the use of an anchor frame subcomponent in connection with delivery, deployment, implantation, or use.

### Interstage

In various examples, the interstage 1300 includes a conduit 1302 that is coupled to an anchor frame subcomponent outlet end and is coupled to a leaflet frame subcomponent inlet end. The conduit 1302 may comprise any suitable material known in the art that is flexible and is operable to be everted as discussed below. By way of example, the conduit 1302 may be a film or fabric, among others.

In various examples, the interstage 1300 further comprises one or more nesting retention elements (not shown), operable to retain the position of the leaflet frame subcomponent 1200 relative to the anchor frame subcomponent 1100 (e.g., nested configuration).

The interstage 1300 is generally any material that is biologically compatible and configured to couple to the anchor frame subcomponent 1100 and the leaflet frame subcomponent 1200. In various examples, the biocompatible material is a film that is not of a biological source and that is sufficiently flexible and strong for the particular purpose, such as a biocompatible polymer. In other examples the interstage 1300 may be of biological tissue (e.g. a dehydrated bovine tissue). In an embodiment, the film comprises a biocompatible polymer (e.g., ePTFE). In some examples, the film is a composite of two or more materials. The film may comprise one or more of a membrane, composite material, or laminate.

In various examples, a prosthetic valve 1001 and its associated delivery system 1002 are configured to facilitate continued valve functionality during the deployment procedure. In various examples, during a prosthetic valve deployment procedure to replace a damaged valve, the damaged valve and valve orifice are temporarily obstructed by the prosthetic valve 1001 and the delivery system 1002. In some instances, such obstructions occur prior to the prosthetic valve 1001 being deployed and becoming operational (e.g., prior to nesting the anchor frame subcomponent 1100 and the leaflet frame subcomponent 1200). Accordingly, in various examples, the prosthetic valves of the present disclosure may additionally include one or more features that are configured to permit fluid to flow through or around the prosthetic valve 1001 during the implantation procedure, prior to the prosthetic valve 1001 becoming fully operational (e.g., prior to nesting the anchor frame subcomponent 1100 and the leaflet frame subcomponent 1200). For example, the prosthetic valve 1001 optionally includes one or more flow enabling features (not shown) formed in the interstage 1300. In some examples, the one or more flow enabling features additionally or alternatively include one or more mechanisms that facilitate unidirectional flow. For instance, in some examples, the flow enabling features are configured as one-way valves.

In some examples, the prosthetic valve 1001 additionally or alternatively includes one or more features that extend between the anchor frame subcomponent 1100 and the leaflet frame subcomponent 1200. For example, the prosthetic valve 1001 optionally includes a plurality of interconnecting struts (not shown) that extend between the anchor frame subcomponent 1100 and the leaflet frame subcomponent 1200. The interconnecting struts are optionally configured to evert along with the interstage 1300 as the leaflet frame subcomponent 1200 is telescoped or nested with the anchor frame subcomponent 1100.

In accordance with some examples, the prosthetic valve 1001 includes one or more nesting retention elements (not shown) in the form of a sinuous (or otherwise shaped) element (e.g., a continuously extending sinuous member) that extends between the anchor frame subcomponent 1100 and the leaflet frame subcomponent 1200 but does not couple directly therewith. Such a sinuous element can help provide stiffening bias to the interstage 1300 and retain the leaflet frame subcomponent 1200 and anchor frame subcomponent 1100 in the nested configuration.

### Anchor Frame Subcomponent

Also as shown in FIG. 7A, the anchor frame subcomponent 1100 includes a compressible, open framework in the form of an anchor frame 1101. The side of the anchor frame 1101 may be at least partially covered, such as with a film or fabric, not shown in FIG. 7B for clarity, suitable for a particular purpose, such as to restrict fluid from passing through the anchor frame 1101, or to encourage tissue ingrowth at the implant site. For illustrative purposes, the following examples are suitable especially for a transcatheter application, but are also suitable for a surgical application.

In accordance with some embodiments, the anchor frame subcomponent 1100 comprises a shape memory material operable to flex under load and retain its original shape when the load is removed, thus allowing the anchor frame subcomponent 1100 to self-expand from a compressed shape to a predetermined larger shape. The anchor frame subcomponent 1100 may comprise the same or different materials as the leaflet frame subcomponent 1200. In accordance with an embodiment, the anchor frame subcomponent 1100 is plastically deformable to be expanded by a balloon. In another embodiment the anchor frame subcomponent 1100 is elastically deformable so as to be self-expanding.

In various embodiments, the prosthetic valve 1001 is configured such that the anchor frame subcomponent 1100 and the leaflet frame subcomponent 1200 can be nested in-situ after the anchor frame subcomponent 1100 and the leaflet frame subcomponent 1200 are deployed at a treatment site in a patient's anatomy. That is, in various embodiments, the prosthetic valve 1001 can be delivered to a treatment region within a patient's anatomy with the anchor frame subcomponent 1100 and the leaflet frame subcomponent 1200 longitudinally offset relative to one another and subsequently nested with one another at the treatment site.

In various examples, the anchor frame subcomponent 1100 is configured to couple to a valve orifice. Accordingly, in various examples, a diameter of the anchor frame subcomponent 1100 (e.g., a diameter of an interior or exterior surface of the anchor frame subcomponent 1100) is sized in accordance with patient anatomy. It will be appreciated that nonlimiting examples of anchor frame subcomponents 1100 can be provided with a diameter (e.g., a diameter of an interior or exterior surface of the anchor frame subcomponent 1100) in a range from about twenty-five (25) millimeters and to about fifty (50) millimeters, depending on a patient's anatomy. However, anchor frame subcomponents 1100 having diameters (e.g., a diameter of an interior or exterior surface of the anchor frame subcomponent 1100) less than twenty-five (25) millimeters and in excess of fifty (50) millimeters are also envisioned and fall within the scope of the present disclosure, depending on patient anatomy.

### Subcomponent Assembly

FIG. 7B shows the leaflet frame subcomponent 1200 and an anchor frame subcomponent 1100 of a prosthetic valve 1001 in a nested configuration, also referred to as the deployed position. Leaflets and any film are not shown for clarity. Any number of leaflets, for example three (3) leaflets 1814 as shown in FIG. 8E, may be coupled to the leaflet frame subcomponent 1200. It is in the nested configuration that the prosthetic valve 1001 remains in the native valve orifice to function as a prosthetic valve. The anchor frame subcomponent 1100 and the leaflet frame subcomponent 1200 are longitudinally offset in a delivery configuration and are generally coaxial relative to one another in a nested configuration. In the nested configuration, the leaflet frame subcomponent 1200, onto which leaflets are coupled, is positioned at least partially within the anchor frame subcomponent 1100.

### Additional Prosthetic Valve Examples

FIGS. 8A through 8C show additional examples of configurations of the prosthetic valve 1001 usable in the transcatheter system 1000, according to some embodiments. These examples are illustrative that any of a variety of prosthetic valve configurations may benefit from the manipulation system 1003. For example, in FIG. 8A, the prosthetic valve 1001 includes a leaflet frame subcomponent 1200, which is the portion of the prosthetic valve 1001 that is disposed at the valve orifice 26, an atrial flange component 1802 extending from one end of the leaflet frame subcomponent 1200, and a plurality of ventricular tethers 1804 extending from another end of the leaflet frame subcomponent 1200. The atrial flange component 1802 may be configured and positioned to prevent migration of the prosthetic valve 1001 into the left or right atrium or into the left or right ventricle. The ventricular tethers 1804 extend from one end of the prosthetic valve 1001 through the heart wall (e.g., the myocardium), and attaches to an anchor portion 1806 positioned on an exterior heart wall (e.g., the epicardium), located opposite from the prosthetic valve 1001. As such, the atrial flange component 1802 and the ventricular tethers 1804 may act together to prevent migration of the prosthetic valve 1001 in opposing directions.

In FIG. 8B, the prosthetic valve 1001 includes the leaflet frame subcomponent 1200 and two or more extension portions 1808 protruding outwardly from the side of the leaflet frame subcomponent 1200. The extension portions 1808 have a plurality of subannular hooks 1810 which act as a stopping mechanism to prevent the prosthetic valve 1001 from migrating into the left or right atrium or into the left or right ventricle, in the absence of an anchoring portion that may be formed in the prosthetic valve 1001. To achieve this, the subannular hooks 1810 may be configured to be directed at a direction against the flow of fluid within the prosthetic valve 1001. The subannular hooks 1810 may then be configured to contact the tissue at or near the valve orifice 26 such that in some examples the subannular hooks 1810 are lodged or otherwise engage the tissue adjacent the valve orifice 26.

In FIG. 8C, the prosthetic valve 1001 includes the leaflet frame subcomponent 1200 and an atrial cage portion 1812 that extends from one end of the leaflet frame subcomponent 1200. The atrial cage portion 1812 is configured to be disposed within the left or right atrium so as to prevent the prosthetic valve 1001 from migrating into the left or right ventricle. In some examples, the atrial cage portion 1812 occupies more volume than the leaflet frame subcomponent 1200. In some examples, the atrial cage portion 1812, when fully expanded, is disposed along an inner surface of the atrium and thus atrial cage portion 1812is disposed along at least 50%, at least 60%, at least 70%, or at least 80% of the inner surface of the left or right atrium.

It is to be understood that, although FIGS. 8A through 8C illustrate various examples of the prosthetic valve 1001 that are disposed in the valve orifice 26 between the left atrium 22 and the left ventricle 18, other examples may include the prosthetic valve 1001 being disposed in a different location within the body, as desired (e.g., in the tricuspid or aortic valves, in the vasculature, or elsewhere).

Although embodiments provided above comprise a framework that supports the leaflets, it is understood and appreciated that the leaflets may not necessarily be supported by a frame. In accordance with some embodiments, the leaflets may be supported by the wall of a solid-walled conduit, the solid-walled conduit being understood to be a frame or support structure. In other embodiments, the leaflets may be constructed as in the tissue valve art that are formed into the desired shape without a frame.

The following non-limiting examples are provided to further illustrate embodiments. It should also be readily appreciated that other leaflet frame designs may be used other than those illustrated in the examples below and accompanying figures.

FIG. 8D is another example of a prosthetic valve 1001 including a configuration similar to that of FIGS. 7A and 7B without an additional anchoring frame. In particular, the leaflet frame subcomponent 1200 may be configured to directly engage the valve orifice and omit the anchor frame subcomponent and interstage of prior examples.

FIG. 8F is an isometric view of a prosthetic valve 1001 in accordance with another embodiment usable with any of the manipulation and delivery systems described herein. As shown, the prosthetic valve 1001 includes an anchoring member, a valve support positioned radially within at least a portion of the anchoring member, and leaflets retained within the valve support.

### Prosthetic Valve: Leaflets

As presented herein, examples of prosthetic valves include flexible leaflets that move in response to changing fluid pressure. The leaflets may be provided as individual components, referred to as leaflets, or may be part of a larger multi-leaflet component referred to as a leaflet construct. It is appreciated that leaflets and leaflet constructs may be constructed in many ways. By way of example, a leaflet may be cut out of a flat sheet or tubular member. By way of another example, a leaflet construct may be cut out of a tubular member or a flat sheet that has been subsequently rolled into a tubular shape. These are but a few examples of forming leaflets or leaflet constructs, which also include, but not limited to, compression and injection molding processes.

For simplicity of discussion, when referring to materials from which leaflets are made, it is appreciated that the same material may also be used to make one or more portions or an entirety of a leaflet construct. Therefore, in this context, the description of leaflet materials applies to options that may be employed for one or more individual leaflets, and also one or more portions of a leaflet construct, as well as for an entirety of the leaflet construct. In the examples that follow, the leaflets that are formed with the leaflet materials described are flexible and are comprised of flexible materials.

Suitable leaflet materials include natural materials (e.g., repurposed tissue, including bovine tissue, porcine tissue, or others), synthetic materials (e.g., biocompatible polymers), and combinations of natural and synthetic materials. Suitable leaflet forming processes include, but are not limited to, casting, molding, extruding, wrapping, coating, imbibing, laminating, combinations thereof and others.

Suitable synthetic leaflet materials include urethanes, silicones (e.g., organopolysiloxanes), copolymers of silicon-urethane, styrene/isobutylene copolymers, polyisobutylene, polyethylene, polyethylene-co-poly(vinyl acetate), polyester copolymers, nylon copolymers, fluorinated hydrocarbon polymers, fluoroelastomers (e.g., copolymers of tetrafluoroethylene and perfluoromethyl vinyl ether (TFE/PMVE copolymer) and (per)fluoroalkylvinylethers (PAVE)), and copolymers and/or mixtures of each of the foregoing and composite materials made therewith. Suitable biocompatible polymers, such as one or more of those described above, may exhibit the physical properties of an elastomer, elastomeric, or non-elastomeric material.

Leaflet materials may include composite materials. Suitable composite leaflet materials include, but are not limited to, one or more membranes combined with one or more polymers. In accordance with some examples, the composite material comprises a membrane (e.g., porous synthetic polymer membrane) by weight in a range of about 10% to about 90%. The one or more polymers may be coatings or layers on the one or more membranes and/or may be imbibed into the one or more membranes (e.g., where the one or more membranes include a microporous structures), for example. Composite materials may include additional or alternative components, such as but not limited to, inorganic fillers, therapeutic agents, radiopaque markers, and others. In some examples, composite leaflet material includes at least one porous synthetic polymer membrane layer having a plurality of pores and/or spaces and a polymer that is an elastomer and/or an elastomeric material filling the pores and/or spaces. In other examples, the composite leaflet material further comprises a layer or coating of elastomer and/or elastomeric material and/or non-elastomeric material on one or both sides of the composite leaflet material.

A membrane that is suitable for use in composite leaflet materials includes, but is not limited to, porous synthetic polymer membranes, such as microporous polyethylene and expanded fluoropolymer membranes such as expanded polytetrafluoroethylene (ePTFE). Such membranes can comprise PTFE homopolymer, blends of PTFE, expandable modified PTFE and/or expanded copolymers of PTFE. As referenced, the membranes may have a microporous structures (e.g., such as ePTFE membranes including a matrix of fibrils defining a plurality of spaces within the matrix).

Suitable polymers of composite leaflet materials include polymers that exhibit elastomer, elastomeric, and/or non-elastomeric material properties. Such polymers may include elastomers and elastomeric materials, such as fluoroelastomers. Examples of suitable polymers include TFE-PMVE copolymers, which may exhibit elastomer, elastomeric, and/or non-elastomeric material properties based on the wt % or mol % of the respective polymers. By way of example of a suitable elastomer, TFE/PMVE copolymer is an elastomer when comprising essentially of between 60 and 20 weight percent tetrafluoroethylene and respectively between 40 and 80 weight percent perfluoromethyl vinyl ether. By way of example of a suitable elastomeric material, TFE/PMVE copolymer is an elastomeric material when comprising essentially of between 67 and 61 weight percent tetrafluoroethylene and respectively between 33 and 39 weight percent perfluoromethyl vinyl ether. By way of example of a suitable non-elastomeric material, TFE/PMVE copolymer is a non-elastomeric material when comprising essentially of between 73 and 68 weight percent tetrafluoroethylene and respectively between 27 and 32 weight percent perfluoromethyl vinyl ether. In the foregoing examples, the TFE and PMVE components of the TFE-PMVE copolymer are presented in wt %. For reference, the wt % of PMVE of 40, 33-39, and 27-32 corresponds to a mol % of 29, 23-28, and 18-22, respectively.

In some examples, the composite leaflet material includes an expanded polytetrafluoroethylene (ePTFE) membrane having been imbibed with TFE-PMVE copolymer comprising from about 60 to about 20 weight percent tetrafluoroethylene and respectively from about 40 to about 80 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces. In other examples the leaflet is an ePTFE membrane having been imbibed with TFE-PMVE copolymer comprising from about 70 to about 61 weight percent tetrafluoroethylene and respectively from about 33 to about 39 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces.

### Tissue Ingrowth Features

In various embodiments, one or more portions of the prosthetic valve 1001 are configured to promote tissue ingrowth. Any portion of the anchor frame subcomponent 1100, leaflet frame subcomponent 1200, interstage 1300, leaflets and/or leaflet construct, or any other feature or portion of the prosthetic valve 1001 may be constructed in a manner that promotes tissue ingrowth, whether over the entire surface(s) or portion(s) thereof. Moreover, such ingrowth may be selective in depth in that growth may by promoted only into the surface of materials, without growing entirely through the material forming the above noted-features. A variety of membranes, films, coatings, fabrics, or other material configurations may be implemented. Some nonlimiting examples of materials that can be applied to portions of the prosthetic valve 1001 to promote tissue ingrowth include properly configured expanded polytetrafluoroethylene (ePTFE), such as an ePTFE membrane and/or polyethylene terephthalate fabric (e.g., Dacron fabric).

In various embodiments, one or more portions of the leaflet frame subcomponent 1200 may be suitable for promoting tissue ingrowth. For example, the leaflet frame 1201 can be wrapped, covered, or otherwise coupled to a cover material suitable for promoting tissue ingrowth. In various examples, such tissue ingrowth promoting materials can be applied to leaflet frame entirely, or to less than all of the leaflet frame as desired. Similarly, one or more portions of the anchor frame subcomponent 1100 may be suitable for promoting tissue ingrowth. For example, the anchor frame 1101 can be wrapped, covered, or otherwise coupled to a cover material suitable for promoting tissue ingrowth. In various examples, such tissue ingrowth promoting materials can be applied to leaflet frame entirely, or to less than all of the leaflet frame 1201 and/or the anchor frame 1101 as desired. For example, suitable materials for promoting tissue ingrowth could be coupled to the inner and/or outer surfaces of the leaflet frame 1201 and/or the anchor frame 1101.

In some embodiments, one or more of the leaflets 1210 may be constructed to encourage tissue ingrowth and proliferation across one or more discrete regions, portions, or sections of one or more of the materials forming the one or more leaflets 1210, or alternatively across an entirety of one or more of the materials forming each of the one or more leaflets 1210. Tissue ingrowth and proliferation may be promoted on an outflow side or surface of one or more of the leaflets 1210, and/or on an inflow side or surface of the leaflets 1210, and/or within one or more materials forming the leaflets.

According to some examples, tissue ingrowth is facilitated by incorporating one or more tissue ingrowth layers into the leaflets 1210 to permit selective growth into one or more surfaces of the one or more leaflets 1210. In some examples, one or more non-ingrowth layers are also incorporated to prevent ingrowth through an entire thickness of the leaflet material, or through unwanted portions of the leaflet. In various examples, material underlying the tissue ingrowth material (e.g., one or more layers) may inhibit tissue ingrowth to limit depth of tissue ingrowth into the leaflet material. Similar concepts may be applied to promote selective tissue ingrowth into the other portions of the prosthetic valve 1001 as referenced above. In other words, any portion of the prosthetic valve 1001 may benefit from incorporation of tissue ingrowth layer(s), as well as one or more non-ingrowth layers as desired.

In some examples, selective promotion of tissue ingrowth into tissue ingrowth layers is facilitated by selectively imbibing, such as with one or more fluoroelastomers, one or more portions of the materials forming the one or more leaflets 1210. Reference to “selectively imbibing” is referring to the act of imbibing a porous material with a filling material at selected portions of the porous material or to a lesser degree leaving a degree of porosity of the porous material. Similar concepts may be applied to promote selective tissue ingrowth into the other portions of the prosthetic valve 1001 as referenced above.

In various embodiments, tissue ingrowth layer materials include expanded fluoropolymer membranes, which may comprise a plurality of spaces within a matrix of fibrils, and are suitable for promoting and supporting the ingrowth of tissue. Other nonlimiting example materials include other biocompatible porous materials such as knit PTFE. However, rather than including a membrane or carrier, in some examples tissue ingrowth layers may be realized in the form of one or more coatings applied to an underlying material.

Though in some examples tissue ingrowth layers include expanded fluoropolymer made from porous ePTFE membranes, it is appreciated that tissue ingrowth layers may be formed from a number of different types of membranes, including other fluoropolymer membranes, and other biocompatible porous materials such as knit PTFE. For instance, suitable expandable fluoropolymers can comprise PTFE homopolymer. In some examples, tissue ingrowth layers can be formed from copolymers of hexafluoropropylene and tetrafluoroethylene, such as fluorinated ethylene propylene (FEP). In some examples, blends of PTFE, expandable modified PTFE and/or expanded copolymers of PTFE can be used. It will thus be appreciated that tissue ingrowth layers may be formed from a variety of different polymeric materials, provided they are biocompatible and possess or are modified to include a suitable microstructure suitable for promoting or supporting tissue ingrowth. In various non-limiting examples, the tissue ingrowth layers may range in thickness from between one micron and four hundred microns, for example, depending on the selected material.

In some examples, polymeric materials for tissue ingrowth layers may include one or more naturally occurring and/or one or more artificially created pores, reliefs, channels, and/or predetermined surface topology, suitable for supporting tissue ingrowth. Other biocompatible porous materials which can be suitable for use forming tissue ingrowth layers include but are not limited to groups of urethanes, fluoropolymers, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

While the above-discussed tissue ingrowth layers may generally include membranes, films, knits, or other structures that are bonded, applied, or otherwise attached to underlying layers, in some examples the tissue ingrowth layers may be applied to underlying layers in the form of one or more coatings. In some such examples, coherent irregular networks are distributed or deposited onto one or more portions, regions, sections, areas, or zones of underlying layers. In some examples, such coherent irregular networks are applied to one or more portions of one or more underlying layers to create a surface texture suitable for supporting the ingrowth and proliferation of tissue, as those of skill will appreciate.

In some examples, coherent irregular networks may be selectively applied to one or more discrete or designated sections, portions, or regions of underlying material. In some such examples, the coherent irregular network is applied to the designated areas by masking or otherwise covering those portions of the underlying material where ingrowth of tissue is undesirable.

In various examples, to achieve layers that promotes or otherwise accommodates ingrowth and proliferation of tissue, expanded fluoropolymer membranes may be selectively imbibed, such as with one or more fluoroelastomers, such that the expanded fluoropolymer membrane includes one or more discrete portions, regions, sections, zones, or areas that are free of or are not otherwise imbibed with the elastomeric filler material (or at least are not filled to the extent that the elastomeric filler material operates to prevent tissue ingrowth).

While the above discussed embodiments and examples include applying tissue ingrowth layers to one or more portions of one or more surfaces of an underlying material, or selectively imbibing one or more portions of one or more sides of a membrane of an underlying material with a filler material, it is appreciated that, in various examples, tissue ingrowth layers may be constructed by both imbibing one or more portions of a membrane forming an underlying material and applying a tissue ingrowth layers to the selectively imbibed underlying material. In various examples, the imbibing material, or filler material, may be varied within tissue ingrowth layers. That is, in some examples, a first portion, area, region, section, or zone of membrane may be imbibed with a first filler material while a second portion, area, region, section, or zone of the membrane may be imbibed with a second filler material.

### Bio-Active Agents

In some embodiment, all or a part of the prosthetic valve, including the leaflets, may be provided with a biologically active (bio-active) agent. Bio-active agents can be coated onto a portion or the entirety of the prosthetic valve, including the leaflet and/or leaflet construct, for controlled release of the agents once the prosthetic valve is implanted.

Such bio-active agents can include, but are not limited to, anti-thrombogenic agents such as, but not limited to, heparin. Bio-active agents can also include, but are not limited to agents such as anti-proliferative/antimitotic agents including natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin) and mitomycin, enzymes (e.g., L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); anti-proliferative/antim itotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine {cladribine}); platinum coordination complexes (e.g., cisplatin and carboplatin), procarbazine, hydroxyurea, mitotane, am inoglutethim ide; hormones (e.g., estrogen); anti-coagulants (e.g., heparin, synthetic heparin salts and other inhibitors of thrombin); anti-platelet agents (e.g., aspirin, clopidogrel, prasugrel, and ticagrelor); vasodilators (e.g., heparin, aspirin); fibrinolytic agents (e.g., plasminogen activator, streptokinase, and urokinase), aspirin, dipyridamole, ticlopidine, clopidogrel, abciximab; antimigratory agents; antisecretory agents (e.g., breveldin); anti-inflammatory agents, such as adrenocortical steroids (e.g., cortisol, cortisone, fludrocortisone, prednisone, prednisolone, 6a-methylprednisolone, triamcinolone, betamethasone, and dexamethasone), non-steroidal agents (e.g., salicylic acid derivatives, such as aspirin); para-aminophenol derivatives (e.g., acetaminophen); indole and indene acetic acids (e.g., indomethacin, sulindac, and etodalac), heteroaryl acetic acids (e.g., tolmetin, diclofenac, and ketorolac), arylpropionic acids (e.g., ibuprofen and derivatives), anthranilic acids (e.g., mefenamic acid and meclofenamic acid), enolic acids (e.g., piroxicam, tenoxicam, phenylbutazone, and oxyphenthatrazone), nabumetone, gold compounds (e.g., auranofin, aurothioglucose, and gold sodium thiomalate); immunosuppressives (e.g., cyclosporine, tacrolimus (FK-506), sirolimus (rapamycin), azathioprine, and mycophenolate mofetil); angiogenic agents (e.g., vascular endothelial growth factor (VEGF)), fibroblast growth factor (FGF); angiotensin receptor blockers; nitric oxide donors; anti-sense oligonucleotides and combinations thereof; cell cycle inhibitors, mTOR inhibitors, growth factor receptor signal transduction kinase inhibitors; retinoids; cyclin/CDK inhibitors; HMG co-enzyme reductase inhibitors (statins); and protease inhibitors.

Although the embodiments herein may be described in connection with various principles and beliefs, the described embodiments should not be bound by theory. For example, embodiments are described herein in connection with prosthetic valves, more specifically cardiac prosthetic valves. However, embodiments within the scope of this disclosure can be applied toward any valve or mechanism of similar structure and/or function. Furthermore, embodiments within the scope of this disclosure can be applied in non-cardiac applications.

The scope of the concepts addressed in this disclosure has been described above both generically and with regard to specific examples. It will be apparent to those skilled in the art that various modifications and variations can be made in the examples without departing from the scope of the disclosure. Likewise, the various components discussed in the examples discussed herein are combinable. Thus, it is intended that the examples cover the modifications and variations of the scope.

1-21. (canceled)

22. A transcatheter system comprising:
a prosthetic valve comprising a first anchor frame subcomponent, a second anchor frame subcomponent, and an interstage disposed between the first anchor frame subcomponent and the second anchor frame subcomponent; and
a constraining sheath configured to be coupled with the prosthetic valve by passing through a portion of the prosthetic valve.

23. The system of claim 22, wherein the first anchor frame subcomponent includes a support structure having a framework, and the constraining sheath is configured to pass through an aperture formed in the framework of the support structure.

24. The system of claim 22, wherein the first anchor frame subcomponent includes a support structure and a cover associated with the support structure, and the constraining sheath is configured to pass through the support structure by penetrating through the cover.

25. The system of claim 22, wherein the interstage includes a cover and defines a conduit, wherein the constraining sheath is configured to penetrate the cover to form an opening in the conduit through which the constraining sheath passes.

26. The system of claim 22, further comprising a manipulation device comprising an elongate portion configured to be slidably received inside the constraining sheath.

27. The system of claim 26, the manipulation device further comprising an engaging portion extending from the elongate portion, the engaging portion being configured to couple to an internal body structure to be manipulated.

28. The system of claim 27, wherein the elongate portion and the engaging portion are integrally formed.

29. The system of claim 27, wherein the engaging portion comprises a pledget anchor and a stop mechanism configured to receive and compress an anterior mitral leaflet therebetween.

30. The system of claim 27, wherein the engaging portion comprises a recurved hook configured to puncture an anterior mitral leaflet.

31. The system of claim 27, wherein the engaging portion includes at least one curved section having a radius of curvature.

32. The system of claim 27, wherein the elongate portion and the engaging portion are formed of an elastic filament material.

33. The system of claim 27, wherein the engaging portion comprises a coupling section, a piercing section, and an anchoring section disposed between the coupling section and the piercing section.

34. The system of claim 33, wherein the coupling section is releasably coupled with the elongate portion of the manipulation device.

35. The system of claim 22, wherein the prosthetic valve is configured to elastically transition from a compact delivery configuration to an expanded deployed configuration.
